SEQUENCE LISTING (66 sequences)

SEQ ID NO: 1 - protein, 92 aa, Homo sapiens
APDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 2 - protein, 92 aa, Artificial Sequence
  human chorionic gonadotropin alpha-subunit with Cys substituted for Gln5
APDVCDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 3 - protein, 92 aa, Artificial Sequence
  human chorionic gonadotropin alpha-subunit with Cys substituted for Leu12
APDVQDCPECTCQENPFFSQPGAPILQCMGCCFSRAYPTPLRSKKTMLVQKNVTSESTCCVAKSYNRVTVMGGFKVENHTACHCSTCYYHKS

SEQ ID NO: 4 - protein, 92 aa, Artificial Sequence
  human chorionic gonadotropin alpha-subunit with Cys substituted for Asn15
APDVQDCPECTLQECPFFSQPGAPILQCMGCCFSRAYPTPLRSKKTMLVQKNVTSESTCCVAKSYNRVTVMGGFKVENHTACHCSTCYYHKS

SEQ ID NO: 5 - protein, 92 aa, Artificial Sequence
  human chorionic gonadotropin alpha-subunit with Cys substituted for Phe17
APDVQDCPECTLQENPCFSQPGAPILQCMGCCFSRAYPTPLRSKKTMLVQKNVTSESTCCVAKSYNRVTVMGGFKVENHTACHCSTCYYHKS

SEQ ID NO: 6 - protein, 92 aa, Artificial Sequence
  human chorionic gonadotropin alpha-subunit with Cys substituted for Leu22
APDVQDCPECTLQENPFFSQPCAPILQCMGCCFSRAYPTPLRSKKTMLVQKNVTSESTCCVAKSYNRVTVMGGFKVENHTACHCSTCYYHKS

SEQ ID NO: 7 - protein, 92 aa, Artificial Sequence
  human chorionic gonadotropin alpha-subunit with Cys substituted for Gln7
APDVQDCPECTLQENPFFSQPGAPILCCMGCCFSRAYPTPLRSKKTMLVQKNVTSESTCCVAKSYNRVTVMGGFKVENHTACHCSTCYYHKS

SEQ ID NO: 8 - protein, 92 aa, Artificial Sequence
  human chorionic gonadotropin alpha-subunit with Cys subsituted for Leu22
APDVQDCPECTCQENPFFSQPCAPILQCMGCCFSRAYPTPLRSKKTMLVQKNVTSESTCCVAKSYNRVTVMGGFKVENHTACHCSTCYYHKS

SEQ ID NO: 9 - protein, 92 aa, Artificial Sequence
  human chorionic gonadotropin alpha-subunit with Cys subsituted for Arg35
APDVQDCPECTLQENPFFSQPGAPILQCMGCCFSCAYPTPLRSKKTMLVQKNVTSESTCCVAKSYNRVTVMGGFKVENHTACHCSTCYYHKS

SEQ ID NO: 10 - protein, 92 aa, Artificial Sequence
  human chorionic gonadotropin alpha-subunit with Cys substituted for Tyr37
APDVQDCPECTLQENPFFSQPGAPILQCMGCCFSRACPTPLRSKKTMLVQKNVTSESTCCVAKSYNRVTVMGGFKVENHTACHCSTCYYHKS

SEQ ID NO: 11 - protein, 92 aa, Artificial Sequence
  human chorionic gonadotropin alpha-subunit with Cys substituted for Pro38
APDVQDCPECTLQENPFFSQPGAPILQCMGCCFSRAYCTPLRSKKTMLVQKNVTSESTCCVAKSYNRVTVMGGFKVENHTACHCSTCYYHKS

SEQ ID NO: 12 - protein, 92 aa, Artificial Sequence
  human chorionic gonadotropin alpha-subunit with Cys substituted for Thr39
APDVQDCPECTLQENPFFSQPGAPILQCMGCCFSRAYPCPLRSKKTMLVQKNVTSESTCCVAKSYNRVTVMGGFKVENHTACHCSTCYYHKS

SEQ ID NO: 13 - protein, 92 aa, Artificial Sequence
  human chorionic gonadotropin alpha-subunit with Cys substituted for Pro40
APDVQDCPECTLQENPFFSQPGAPILQCMGCCFSRAYPTCLRSKKTMLVQKNVTSESTCCVAKSYNRVTVMGGFKVENHTACHCSTCYYHKS

SEQ ID NO: 14 - protein, 92 aa, Artificial Sequence
  human chorionic gonadotropin alpha-subunit with Cys substituted for Leu41
APDVQDCPECTLQENPFFSQPGAPILQCMGCCFSRAYPTPCRSKKTMLVQKNVTSESTCCVAKSYNRVTVMGGFKVENHTACHCSTCYYHKS

SEQ ID NO: 15 - protein, 92 aa, Artificial Sequence
  human chorionic gonadotropin alpha-subunit with Cys substituted for Arg42
APDVQDCPECTLQENPFFSQPGAPILQCMGCCFSRAYPTPLCSKKTMLVQKNVTSESTCCVAKSYNRVTVMGGFKVENHTACHCSTCYYHKS

SEQ ID NO: 16 - protein, 92 aa, Artificial Sequence
  human chorionic gonadotropin alpha-subunit with Cys substituted for Ser43
APDVQDCPECTLQENPFFSQPGAPILQCMGCCFSRAYPTPLRCKKTMLVQKNVTSESTCCVAKSYNRVTVMGGFKVENHTACHCSTCYYHKS

SEQ ID NO: 17 - protein, 92 aa, Artificial Sequence
  human chorionic gonadotropin alpha-subunit with Cys substituted for Lys44
APDVQDCPECTLQENPFFSQPGAPILQCMGCCFSRAYPTPLRSCKTMLVQKNVTSESTCCVAKSYNRVTVMGGFKVENHTACHCSTCYYHKS

SEQ ID NO: 18 - protein, 92 aa, Artificial Sequence
  human chorionic gonadotropin alpha-subunit with Cys substituted for Lys45
APDVQDCPECTLQENPFFSQPGAPILQCMGCCFSRAYPTPLRSKCTMLVQKNVTSESTCCVAKSYNRVTVMGGFKVENHTACHCSTCYYHKS

SEQ ID NO: 19 - protein, 92 aa, Artificial Sequence
  human chorionic gonadotropin alpha subunit with Cys substituted for Thr46
APDVQDCPECTLQENPFFSQPGAPILQCMGCCFSRAYPTPLRSKKCMLVQKNVTSESTCCVAKSYNRVTVMGGFKVENHTACHCSTCYYHKS

SEQ ID NO: 20 - protein, 92 aa, Artificial Sequence
  human chorionic gonadotropin alpha-subunit with Cys substituted for Met47
APDVQDCPECTLQENPFFSQPGAPILQCMGCCFSRAYPTPLRSKKTCLVQKNVTSESTCCVAKSYNRVTVMGGFKVENHTACHCSTCYYHKS

SEQ ID NO: 21 - protein, 92 aa, Artificial Sequence
  human chorionic gonadotropin alpha-subunit with Cys substituted for Leu48
APDVQDCPECTLQENPFFSQPGAPILQCMGCCFSRAYPTPLRSKKTMCVQKNVTSESTCCVAKSYNRVTVMGGFKVENHTACHCSTCYYHKS

SEQ ID NO: 22 - protein, 92 aa, Artificial Sequence
  human chorionic gonadotropin alpha-subunit with Cys substituted for Val49
APDVQDCPECTLQENPFFSQPGAPILQCMGCCFSRAYPTPLRSKKTMLCQKNVTSESTCCVAKSYNRVTVMGGFKVENHTACHCSTCYYHKS

SEQ ID NO: 23 - protein, 92 aa, Artificial Sequence
  human chorionic gonadotropin alpha-subunit with Cys substituted for Gln50
APDVQDCPECTLQENPFFSQPGAPILQCMGCCFSRAYPTPLRSKKTMLVCKNVTSESTCCVAKSYNRVTVMGGFKVENHTACHCSTCYYHKS

SEQ ID NO: 24 - protein, 92 aa, Artificial Sequence
  human chorionic gonadotropin alpha-subunit with Cys substituted for Lys51
APDVQDCPECTLQENPFFSQPGAPILQCMGCCFSRAYPTPLRSKKTMLVQCNVTSESTCCVAKSYNRVTVMGGFKVENHTACHCSTCYYHKS

SEQ ID NO: 25 - protein, 92 aa, Artificial Sequence
  human chorionic gonadotropin alpha-subunit with Cys substituted for Asn52
APDVQDCPECTLQENPFFSQPGAPILQCMGCCFSRAYPTPLRSKKTMLVQKCVTSESTCCVAKSYNRVTVMGGFKVENHTACHCSTCYYHKS

SEQ ID NO: 26 - protein, 92 aa, Artificial Sequence
  human chorionic gonadotropin alpha-subunit with Cys substituted for Val53
APDVQDCPECTLQENPFFSQPGAPILQCMGCCFSRAYPTPLRSKKTMLVQKNCTSESTCCVAKSYNRVTVMGGFKVENHTACHCSTCYYHKS

SEQ ID NO: 27 - protein, 92 aa, Artificial Sequence
  human chorionic gonadotropin alpha-subunit with Cys substituted for Glu56
APDVQDCPECTLQENPFFSQPGAPILQCMGCCFSRAYPTPLRSKKTMLVQKNVTSCSTCCVAKSYNRVTVMGGFKVENHTACHCSTCYYHKS

SEQ ID NO: 28 - protein, 92 aa, Artificial Sequence
  human chorionic gonadotropin alpha-subunit with Cys substituted for Ser64
APDVQDCPECTLQENPFFSQPGAPILQCMGCCFSRAYPTPLRSKKTMLVQKNVTSESTCCVACCYNRVTVMGGFKVENHTACHCSTCYYHKS

SEQ ID NO: 29 - protein, 92 aa, Artificial Sequence
  human chorionic gonadotropin alpha-subunit with Cys substituted for Val76
APDVQDCPECTLQENPFFSQPGAPILQCMGCCFSRAYPTPLRSKKTMLVQKNVTSESTCCVAKSYNRVTVMGGFKCENHTACHCSTCYYHKS

SEQ ID NO: 30 - protein, 92 aa, Artificial Sequence
  human chorionic gonadotropin alpha-subunit with Cys substituted for Thr86
APDVQDCPECTLQENPFFSQPGAPILQCMGCCFSRAYPTPLRSKKTMLVQKNVTSESTCCVAKSYNRVTVMGGFKVENHTACHCSCCYYHKS

SEQ ID NO: 31 - protein, 92 aa, Artificial Sequence
  human chorionic gonadotropin alpha-subunit with Cys substituted for Tyr88
APDVQDCPECTLQENPFFSQPGAPILQCMGCCFSRAYPTPLRSKKTMLVQKNVTSESTCCVAKSYNRVTVMGGFKVENHTACHCSTCCYHKS

SEQ ID NO: 32 - protein, 92 aa, Artificial Sequence
  human chorionic gonadotropin alpha-subunit with Cys substituted for Leu89
APDVQDCPECTLQENPFFSQPGAPILQCMGCCFSRAYPTPLRSKKTMLVQKNVTSESTCCVAKSYNRVTVMGGFKVENHTACHCSTCYCHKS

SEQ ID NO: 33 - protein, 92 aa, Artificial Sequence
  human chorionic gonadotropin alpha-subunit with Cys substituted for His90
APDVQDCPECTLQENPFFSQPGAPILQCMGCCFSRAYPTPLRSKKTMLVQKNVTSESTCCVAKSYNRVTVMGGFKVENHTACHCSTCYYCKS

SEQ ID NO: 34 - protein, 92 aa, Artificial Sequence
  human chorionic gonadotropin alpha-subunit with Cys substituted for Lys91
APDVQDCPECTLQENPFFSQPGAPILQCMGCCFSRAYPTPLRSKKTMLVQKNVTSESTCCVAKSYNRVTVMGGFKVENHTACHCSTCYYHCS

SEQ ID NO: 35 - protein, 92 aa, Artificial Sequence
  human chorionic gonadotropin alpha-subunit with Cys substituted for Ser92
APDVQDCPECTLQENPFFSQPGAPILQCMGCCFSRAYPTPLRSKKTMLVQKNVTSESTCCVAKSYNRVTVMGGFKVENHTACHCSTCYYHKC

SEQ ID NO: 36 - protein, 145 aa, Homo sapiens
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 37 - protein, 145 aa, Artificial Sequence
  human chorionic gonadotropin beta-subunit with Cys substituted for Ser138
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPCDTPILPQ

SEQ ID NO: 38 - protein, 145 aa, Artificial Sequence
  human chorionic gonadotropin beta-subunit residues 101- 114 were replaced with their hFSH beta-subunit counterparts, namely hFSH beta-subunit residues 95-108
SKEPLRPRCRPINATLAVEKEGCPVCITVNTTICAGYCPTMTRVLQGVLPALPQVVCNYRDVRFESIRLPGCPRGVPNVVSYAVALSCQCALCRRSTTDCTVRGLGPSYCSFGEFQDSSSSKAPPPSLPSPSRLPGPSDTPILPQ

SEQ ID NO: 39 - protein, 145 aa, Artificial Sequence
  human chorionic gonadotropin beta-subunit residues 101- 114 were replaced with their hFSH beta-subunit counterparts, namely hFSH beta-subunit residues 95-108, and Serine38 in the beta-subunit carboxyterminus of this analog was replaced with Cys
SKEPLRPRCRPINATLAVEKEGCPVCITVNTTICAGYCPTMTRVLQGVLPALPQVVCNYRDVRFESIRLPGCPRGVPNVVSYAVALSCQCALCRRSTTDCTVRGLGPSYCSFGEFQDSSSSKAPPPSLPSPSRLPGPCDTPILPQ

SEQ ID NO: 40 - protein, 111 aa, Homo sapiens
NSCELTNITIAVEKEGCGFCITINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 41 - protein, 139 aa, Artificial Sequence
  hFSH beta-subunit analog lacking the leader peptide of hFSH beta-subunit with hFSH residues 1-108 and human chorionic gonadotropin residues 115-145 in tandem
NSCELTNITIAVEKEGCGFCITINTTWCAGYCYTRDLVYKDPARPKIQKTCTFKELVYETVRVPGCAHHADSLYTYPVATQCHCGKCDSDSTDCTVRGLGPSYCSFGEFQDSSSSKAPPPSLPSPSRLPGPSDTPILPQ

SEQ ID NO: 42 - protein, 137 aa, Artificial Sequence
  hFSH beta-subunit analog lacking the leader peptide of hFSH beta-subunit with hFSH residues 1-108 and human chorionic gonadotropin residues 115-145 in tandem and with Ser132 replaced with Cys
NSCELTNITIAVEKEGCGFCITINTTWCAGYCYTRDLVYKDPARPKIQKTCTFKELVYETVRVPGCAHHADSLYTYPVATQCHCGKCDSDSTDCTVRGLGPSYCSFGEFQDSSSSKAPPPSLPSPSRLPGPCDTPIL

SEQ ID NO: 43 - protein, 401 aa, Artificial Sequence [sequence omitted]

SEQ ID NO: 44 - protein, 408 aa, Artificial Sequence
  human chorionic gonadotropinbeta, S138C-betaLA(long), beta-lactamase fused to the carboxy terminal end of human chorionic gonadotropinb,S138C
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPCDTPILPQHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 45 - protein, 125 aa, Artificial Sequence
  human chorionic gonadotropinbeta,delta116-135, S138C
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFGPCDTPILPQ

SEQ ID NO: 46 - protein, 130 aa, Artificial Sequence
  human chorionic gonadotropinbeta,delta121-135, S138C
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSGPCDTPILPQ

SEQ ID NO: 47 - protein, 136 aa, Artificial Sequence
  human chorionic gonadotropinbeta,delta126-135, S138C
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPGPCDTPILPQ

SEQ ID NO: 48 - protein, 140 aa, Artificial Sequence
  human chorionic gonadotropinbeta,delta131-135, S138C
SKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSGPCDTPILPQ

SEQ ID NO: 49 - protein, 92 aa, Artificial Sequence
  human chorionic gonadotropin alpha-subunit, Lys91 replaced with Glu
APDVQDCPECTLQENPFFSQPGAPILQCMGCCFSRAYPTPLRSKKTMLVQKNVTSESTCCVAKSYNRVTVMGGFKVENHTACHCSTCYYHES

SEQ ID NO: 50 - protein, 92 aa, Artificial Sequence
  human chorionic gonadotropin alpha-subunit loop 2, Lys91 replaced with Met
APDVQDCPECTLQENPFFSQPGAPILQCMGCCFSRAYPTPLRSKKTMLVQKNVTSESTCCVAKSYNRVTVMGGFKVENHTACHCSTCYYHMS

SEQ ID NO: 51 - protein, 92 aa, Artificial Sequence
  human chorionic gonadotropin alpha-subunit loop 2, Lys44 replaced with Ala
APDVQDCPECTLQENPFFSQPGAPILQCMGCCFSRAYPTPLRSAKTMLVQKNVTSESTCCVAKSYNRVTVMGGFKVENHTACHCSTCYYHKS

SEQ ID NO: 52 - protein, 92 aa, Artificial Sequence
  human chorionic gonadotropin alpha-subunit loop 2, Lys44 replaced with Glu and Lys45 replaced with Gln
APDVQDCPECTLQENPFFSQPGAPILQCMGCCFSRAYPTPLRSEQTMLVQKNVTSESTCCVAKSYNRVTVMGGFKVENHTACHCSTCYYHKS

SEQ ID NO: 53 - protein, 92 aa, Artificial Sequence
  human chorionic gonadotropin alpha-subunit loop 2, Lys44 replaced with Arg
APDVQDCPECTLQENPFFSQPGAPILQCMGCCFSRAYPTPLRSRKTMLVQKNVTSESTCCVAKSYNRVTVMGGFKVENHTACHCSTCYYHKS

SEQ ID NO: 54 - protein, 139 aa, Artificial Sequence
  human chorionic gonadotropin analog - beta101-14 5,alpha, residues 3-100 deleted from  human chorionic gonadotropin beta-subunit with alpha-subunit fused to the end of the remaining beta-subunit
SKGGPKDHPLTCDDPRFQDSSSSKAPPPSLPSPSRLPGPSDTPILPQAPDVQDCPECTLQENPFFSQPGAPILQCMGCCFSRAYPTPLRSKKTMLVQKNVTSESTCCVAKSYNRVTVMGGFKVENHTACHCSTCYYHKS

SEQ ID NO: 55 - protein, 31 aa, Homo sapiens
FQDSSSSKAPPPSLPSPSRLPGPSTDPILPG

SEQ ID NO: 56 - protein, 37 aa, Artificial Sequence
  Xaa may be amino acids 1-10, 17-26 and 28-37 of tailportion: Xaa is any amino acid in the tail portion and some of the Xaa may be missing from the tail portion
XXXXXXXXXXDDDDKSXXXXXXXXXXCXXXXXXXXXX

SEQ ID NO: 57 - protein, 107 aa, Artificial Sequence
  An human chorionic gonadotropin truncated (-subunit analog fused to the human chorionic gonadotropin alpha-carboxyterminus
APDVQDCPECTLQENPFFSQPGAPILQCMGCCFSRAYPTPLRSKKTMLVQKNVTSESTCCVAKSYNRVTVMGGFKVENHTACHCSTCYYHKSDDPRFGPCDTPILPQ

SEQ ID NO: 58 - protein, 145 aa, Artificial Sequence
  human chorionic gonadotropin beta-subunit with Cys substituted for Arg94
SKEPLRPRCRPINATLAVEKEGCPVCITVNTTICAGYCPTMTRVLQGVLPALPQVVCNYRDVRFESIRLPGCPRGVPNVVSYAVALSCQCALCCRSTTDCGGPKDHPLTCDDPRFQDSSSSKAPPPSLPSPSRLPGPSDTPILPQ

SEQ ID NO: 59 - protein, 145 aa, Artificial Sequence
  human chorionic gonadotropin beta-subunit with Cys substituted for Arg95
SKEPLRPRCRPINATLAVEKEGCPVCITVNTTICAGYCPTMTRVLQGVLPALPQVVCNYRDVRFESIRLPGCPRGVPNVVSYAVALSCQCALCRCSTTDCGGPKDHPLTCDDPRFQDSSSSKAPPPSLPSPSRLPGPSDTPILPQ

SEQ ID NO: 60 - protein, 145 aa, Artificial Sequence
  human chorionic gonadotropin beta-subunit with Cys substituted for Ser96
SKEPLRPRCRPINATLAVEKEGCPVCITVNTTICAGYCPTMTRVLQGVLPALPQVVCNYRDVRFESIRLPGCPRGVPNVVSYAVALSCQCALCRRCTTDCGGPKDHPLTCDDPRFQDSSSSKAPPPSLPSPSRLPGPSDTPILPQ

SEQ ID NO: 61 - protein, 145 aa, Artificial Sequence
  human chorionic gonadotropin beta-subunit with Cys substituted for Thr97
SKEPLRPRCRPINATLAVEKEGCPVCITVNTTICAGYCPTMTRVLQGVLPALPQVVCNYRDVRFESIRLPGCPRGVPNVVSYAVALSCQCALCRRSCTDCGGPKDHPLTCDDPRFQDSSSSKAPPPSLPSPSRLPGPSDTPILPQ

SEQ ID NO: 62 - protein, 145 aa, Artificial Sequence
  human chorionic gonadotropin beta-subunit with Cys substituted for Thr98
SKEPLRPRCRPINATLAVEKEGCPVCITVNTTICAGYCPTMTRVLQGVLPALPQVVCNYRDVRFESIRLPGCPRGVPNVVSYAVALSCQCALCRRSTCDCGGPKDHPLTCDDPRFQDSSSSKAPPPSLPSPSRLPGPSDTPILPQ

SEQ ID NO: 63 - protein, 145 aa, Artificial Sequence
  human chorionic gonadotropin beta-subunit with Cys substituted for Asp99
SKEPLRPRCRPINATLAVEKEGCPVCITVNTTICAGYCPTMTRVLQGVLPALPQVVCNYRDVRFESIRLPGCPRGVPNVVSYAVALSCQCALCRRSTTCCGGPKDHPLTCDDPRFQDSSSSKAPPPSLPSPSRLPGPSDTPILPQ

SEQ ID NO: 64 - protein, 95 aa, Artificial Sequence
  An human chorionic gonadotropin alpha-subunit analog with Gly-Gly-Cys at its carboxyterminus
APDVQDCPECTLQENPFFSQPGAPILQCMGCCFSRAYPTPLRSKKTMLVQKNVTSESTCCVAKSYNRVTVMGGFKVENHTACHCSTCYYHKSGGC

SEQ ID NO: 65 - protein, 92 aa, Artificial Sequence
  An human chorionic gonadotropin alpha-subunit analog with Asp in place of Asn52 and Cys in place of Ser92
APDVQDCPECTLQENPFFSQPGAPILQCMGCCFSRAYPTPLRSKKTMLVQKDVTSESTCCVAKSYNRVTVMGGFKVENHTACHCSTCYYHKC

SEQ ID NO: 66 - protein, 145 aa, Artificial Sequence
  human chorionic gonadotropin beta-subunit with Cys substituted for Ser96 and hFSH beta-subunit residues 95-108 for human chorionic gonadotropin beta-subunit residues 101-108
SKEPLRPRCRPINATLAVEKEGCPVCITVNTTICAGYCPTMTRVLQGVLPALPQVVCNYRDVRFESIRLPGCPRGVPNVVSYAVALSCQCALCRRCTTDCTVRGLGPSYCSFGEFQDSSSSKAPPPSLPSPSRLPGPSDTPILPQ

(19) United States
(12) Patent Application Publication
(10) Pub. No.: US 2006/0177894 A1
(43) Pub. Date: Aug. 10, 2006

(54) Protein knobs

(76) Inventors: William R. Moyle, Piscatawy, NJ (US); Yongna Xing, Edison, NJ (US)

Correspondence Address: SERONO RESEARCH INSTITUTE, ONE TECHNOLOGY PLACE, ROCKLAND, MA, 02370, US

(21) Appl. No.: 10/797,553

(22) Filed: Mar. 10, 2004

(30) Foreign Application Priority Data
Nov. 8, 2002 (WO) PCT/US02/35914

(51) Int. Cl. C07K 14/72; C07H 21/04; C12P 21/06

(52) U.S. Cl. 435/69.1; 435/320.1; 435/325; 530/350; 536/23.5

(57) ABSTRACT

Compositions containing a knob attached to a specific site of a protein and methods of producing and using these compositions are disclosed. The compositions comprise a knob, a tail portion and a protein portion. The protein portion contains a substituted cysteine residue at the desired location of labeling. The tail portion is located at the terminal end of the protein portion. The knob is linked to the end of the tail portion and contains a cysteine residue. The substituted cysteine residue on the protein portion and the cysteine residue on the knob form a disulfide bond, thereby tagging the protein portion at the desired site with the knob.

### GOVERNMENT INTEREST

This work was supported by National Institutes of Health Grants NICHD HD14907 and NICHD HD38547. This invention was made with United States of America government support. The government may own certain rights in the present invention.

### FIELD OF THE INVENTION

The present invention relates to the field of protein tagging.

### INTRODUCTION

Methods for tagging proteins for use in analyzing protein-protein interactions or in protein purification currently involve fusing a tag to the carboxy- or amino-terminal ends of the protein or inserting residues into a loop of the protein. Each of these methods has its limitations. For example, it may be desirable to tag a protein, whether it be with a dye molecule or a protein, at specific locations on the protein, rather than being limited to attaching molecules on the ends of the protein. These include studies designed to probe the interactions of proteins with macromolecules as is the case of hCG with its receptor. In addition, where the terminal ends of a protein are involved in the protein's function, tagging the ends of the protein may not be desirable. With respect to inserting a tag into the loop of a protein, this method is limited because the size of the tag usually must be relatively small (a few residues), unless the tag is inserted between protein domains. There are occasions when it would be desirable to attach various sized probes onto the surface of a protein. Further, modifications involving the use of a cysteine residue are also difficult since the protein may contain other cysteine residues which will require protection or the cysteine may become blocked and protein denaturation may occur when removing the blocking residue. As a result, the tagged proteins produced by these methods have limited use and application in the industry.

For example, efforts to identify portions of human choriogonadotropin (hCG) that contact the lutropin receptor (LHR) have been stymied by the complex structure of the hormone and the likelihood that it interacts with the receptor at multiple sites. The crystal structure of human choriogonadotropin (hCG) revealed that a strand of its β-subunit surrounds the α-subunit like a ‘seatbelt’ (Lapthorn, A. J., Harris, D. C., Littlejohn, A., Lustbader, J. W., Canfield, R. E, Machin, K. J., Morgan, F. J. & Isaacs, N. W (1994) Nature 369, 455-461; Wu, H., Lustbader, J. W., Liu, Y., Canfield, R. E. & Hendrickson, W. A. (1994) Structure 2, 545-558.) Unlike most dimeric proteins that are stabilized entirely by intersubunit contacts, hCG appears to be secured largely by its seatbelt; elimination of the disulfide that ‘latches’ the carboxyterminal end of the seatbelt to Cys26 in the β-subunit core was found to disrupt hCG secretion (Suganuma, N., Matzuk, M. M. & Boime, I. (1989) J. Biol Chem. 264, 19302-19307), presumably by destabilizing the heterodimer. The evolutionary advantages of this unusual structural arrangement remain unknown and may reflect the finding that it permits movements of the subunits within the heterodimer, a phenomenon detected during the binding of some hCG analogs to FSH receptors, (Wang, Y. H., Bemard, M. P. & Moyle, W. R. (2000) Mol. Cell. Endocrinol. 170, 67-77.)

The receptors for all three classes of glycoprotein hormones, including hCG, are coupled to G-proteins and have large extracellular domains containing multiple leucine-rich repeats, (Segaloff, D. L. & Ascoli, M. (1993) Endocr. Rev. 14, 324-347.) The later finding suggests the extracellular domain may be horseshoe-shaped, similar to portions of other leucine-rich repeat proteins, (Kobe, B. & Deisenhofer, J. (1993) Nature 366, 751-756.) Two regions of the extracellular domain appear to contribute to ligand binding affinity and specificity. The affinity of hCG for alternately-spliced and truncated LHR analogs is similar to that for the intact receptor, suggesting that residues in the aminoterminal two-thirds of the extracellular domain form the high affinity ligand binding site, (Braun, T., Schofield, P. R. & Sprengel, R. (1991) EMBO. J. 10, 1885-1890; Thomas, D., Rozell, T. G., Liu, X & Segaloff, D. L. (1996) Mol. Endocrinol. 10, 760-768.) Residues in the carboxyterminal fifth of the human LHR extracellular domain interfere with the binding of non-human mammalian lutropins, a finding that indicates contacts between this portion of the hormone and receptor are primarily steric in nature, (Bemard, M. P., Myers, R. V. & Moyle, W. R. (1998) Biochem. J. 335, 611-617.).

The surfaces of hCG, hFSH, and hTSH most likely to contact the LHR, FSHR, and TSHR remain debated. The carboxyterminal end of the α-subunit, which is adjacent to a portion of the seatbelt in the heterodimer, (Wu, H., Lustbader, J. W, Liu, Y., Canfield, R. E. & Hendrickson, W A. (1994) Structure 2, 545-558), has been found to influence the affinity of all the glycoprotein hormones for their receptors, (Lapthom, A. J., Hards, D. C., Littlejohn, A., Lustbader, J. W., Canfield, R. E., Machin, K. J., Morgan, F. J. & Isaacs, N. W. (1994) Nature 369, 455-461; Bemard, M. P., Myers, R. V. & Moyle, W. R. (1998) Biochem. J. 335, 611-617) and was proposed to be a receptor contact more than 25 years ago, (Lapthom, A. J., Hards, D. C., Littlejohn, A., Lustbader, J. W., Canfield, R. E, Machin, K. J., Morgan, F. J. & Isaacs, N. W. (1994) Nature 369, 455461.) Together with data on the structure and function of the hormone, these observations have led to views of the hormone-receptor complex that differ radically, (Moyle, W. R., Campbell, R. K, Rao, S. N. V., Ayad, N. G., Bemard, M. P., Han, Y. & Wang, Y (1995) J. Biol. Chem. 270, 20020-20031), ranging from one in which the hormone contacts the concave surface of the receptor extracellular domain, (Jiang, X, Dreano, M., Buckler, D. R., Cheng, S., Ythier, A., Wu, H., Hendrickson, W. A., Tayar, N. E. & el Tayar, N. (1995) Structure 3, 1341-1353), to one in which it contacts its rim, (Moyle, W. R., Campbell, R. K., Rao, S. N. V., Ayad, N. G., Bernard, M. P., Han, Y & Wang, Y (1995) J. Biol. Chem. 270, 20020-20031.) All views of the hCG-LHR complex suggest that portions of α-subunit loop 2 face the receptor, but it remains to be determined if this portion of the hormone participates in receptor contacts. Mutation of this loop has been reported to reduce the activity of hCG, (Peng, K. C., Bousfield, G. R., Puett, D. & Ward, D. N. (1996) Journal of Protein Chemistry 15, 547-552; Xia, H., Chen, F. & Puett, D. (1994) Endocrinol. 134, 1768-1770), suggesting that it may make essential LHR contacts.

Protein-receptor interactions are key in understanding the function and regulation of cell behavior. A deep understanding of protein-receptor interactions are necessary for many endeavors, such as the design of new pharmaceutical drugs. Currently, however, there are many limitations to understanding protein-receptor interactions. It is helpful to understand both the structure of the protein's conformation and the protein's function, as well as how the structure of the protein interacts with the receptor (which is also commonly a protein). Through knowledge and experience, one aspect of a protein can sometimes be extrapolated if other aspects are known. Protein-receptor interactions can be modeled on a computer, but modeling is a complex task, especially because molecules are flexible and adopt a number of conformations that are of a similar energy. The modeling of the protein-receptor binding process is also difficult because the characteristics of the receptor, the ligand, and the solvent need to be considered.

In addition to modeling, another approach to understanding protein-receptor interactions is to make changes to the structure of the protein, structure of the receptor, or conditions under which they interact in an experimental setting and measure the effect of the change on the binding, and therefore, on the function of the protein or lack thereof. These experimental techniques also have limitations because of the difficulty in manipulating the protein or receptor and limitations in ability to measure changes. Therefore, an invention that could improve the accuracy of experimental data by consistently causing protein-receptor interaction or consistently inhibiting protein-receptor interaction would be an improvement in the art because it would allow a determination of the function or lack of function caused thereby.

In principle, identification of the portions of the hormone and receptor that contact each other should be readily elucidated by using site-directed mutagenesis. Unfortunately, mutations of the α-subunit carboxyterminus and β-subunit seatbelt alter the positions of the subunits within the heterodimer, (Jiang, X., Dreano, M., Buckler, D. R., Cheng, S., Ythier, A., Wu, H., Hendrickson, W. A., Tayar, N. E. & el Tayar, N. (1995) Structure 3, 1341-1353; Pierce, J. G. & Parsons, T. F. (1981) Annu. Rev. Biochem. 50, 465-495), and have made it difficult to interpret the influence of these key parts of the hormone on its function. Further, mutations that change hCG activity may do so by altering key LHR contacts, shifting the positions of its subunits, or both. Indeed, mutations to the α-subunit carboxyterminus, (Pierce, J. G. & Parsons, T. F. (1981) Annu. Rev. Biochem. 50, 465-495, Chen, F., Wang, Y. & Puett, D. (1992) Mol. Endocrinol. 6, 914-919, and the seatbelt, Campbell, R. K., Dean Emig, D. M. & Moyle, W. R. (1991) Proc. Natl. Acad. Sci. (U.S.A.) 88, 760-764; Campbell, R. K., Bergert, E. R., Wang, Y., Morris, J. C. & Moyle, W. R. (1997) Nature Biotech. 15, 439-443; Grossmann, M., Szkudlinski, M. W., Wong, R., Dias, J. A., Ji, T. H. & Weintraub, B. D. (1997) J. Biol. Chem. 272, 15532-15540; Lindau-Shepard, B., Roth, K. E. & Dias, J. A. (1994) Endocrinol. 135, 1235-1240), which have large influences on hormone-receptor interactions, also alter the positions of the subunits in the heterodimer, (Wang, Y. H., Bemard, M. P. & Moyle, W. R. (2000) Mol. Cell. Endocrinol. 170, 67-77; Moyle, W. R., Campbell, R. K., Rao, S. N. V., Ayad, N. G., Bemard, M. P., Han, Y. & Wang, Y. (1995) J. Biol. Chem. 270, 20020-20031.) An alternative method for identifying which portions of the protein and receptor interact involves identifying the residues that do not contact the receptor. These residues can be identified with much more certainty. However, it has been difficult to obtain probes for use in identification of these residues. Accordingly, improved methods for producing probes for use in analysis of protein interaction are necessary.

In addition to its use in analysis of protein interactions, probes or proteins tagged at a specific site on the molecule provide invaluable research tools. However as previously described, current methods involve tagginging or attaching a probe to the terminal ends of the molecule, limiting their use. Other methods involve complicated procedures requiring various reactions and protecting groups to produce the site specific modification without modifying other amino acids and the structure of the functional protein. Accordingly, improved methods for site specific tagginging are desired.

Protein purification is frequently a necessary, yet somewhat onerous, process. Purified proteins may be a required intermediate product of a scientific experiment or may be an end product. Protein purity is often critical to experimental and therapeutic success. In some cases, where injectable proteins are scrutinized by the Food and Drug Administration, any contaminants must be removed or proven harmless. In addition to purity, proteins must retain their biological activity.

There are a number methods of protein purification currently known in the art, although all methods have some limitations. Size-exclusion chromatography is helpful for gross separations, but is not an exacting method and requires that samples be concentrated. Gel electrophoresis allows for precise separation of a protein in a mixture, but is practical only for small samples. Affinity chromatography is a useful method, but typically requires that some of the initial contaminants have already been filtered. Accordingly, an invention that could aid in precise separation of proteins with few steps would be a significant improvement in the art, particularly if the invention could be scaled up to purify larger volumes without sacrificing purity.

In addition to these uses, knobs can be used to ‘cover’ specific surfaces on proteins. This type of use has applications in the design of prodrugs that can be used to target tumors or other unwanted tissues such as those found in the ovaries of infertile women diagnosed with polycystic ovary syndrome. Thus, attachment of a knob to near the active site of a toxin or toxic enzyme would permit the toxic enzyme to be used in patients. Once the toxin or enzyme reached the tissue that contained an enzyme capable of cleaving the linker that attached the knob, toxic or enzymatic activity would be restored. This strategy is expected to reduce the presence of undesirable side-effects that may otherwise limit the usefulness of the toxin or enzyme. In the same way, knobs could be used to block the activities of agents such as PTEN that promote apoptosis.

### SUMMARY OF THE INVENTION

The present invention relates to compositions comprising proteins tagged at a specific site with a knob and methods for making and using these compositions. A knob refers a tag for the protein which can be customized for a specified use.

In one aspect of the invention, proteins tagged with a knob at a specific site in the protein are disclosed. The site specific tagged protein contains a knob, a tail portion, and a protein portion. The knob comprises the tag or probe aspect of the protein and has a cysteine residue. The tail portion is located between the knob portion and the protein portion. The protein portion has a cysteine residue substituted for the native amino acid at the desired site of tagging. The cysteine residues of the knob and protein portion form a disulfide bond. In a further aspect of the invention, the tail portion may contain a protease or other cleavage site.

In another aspect of the invention, methods for producing proteins site specifically tagged with knobs are disclosed. These methods involve selecting a desired protein for tagging, locating a specific site on the desired protein for tagging, producing the desired protein, wherein a cysteine residue is substituted at the specific site for tagging in the desired protein, and wherein the desired protein further comprises a tail portion at one end of the protein and a knob at the end of the tail portion, wherein the knob contains a cysteine residue, and wherein the cysteine residue in the knob forms a disulfide bond with the cysteine residue in the protein portion. In a related aspect of the invention, methods for attaching knobs to hCG at specified sites are disclosed. These methods involve inserting constructs capable of expressing native hCGβ or hCGβ-S138C, and native hCGα or hCGα-cysteine substituted analogs into a cell for co-expression, and fusing a knob to residue 140 or 145 of hCGβ.

In another aspect of the invention, methods for protein purification that employ the site specific tagged proteins of this invention are disclosed. These methods involve inserting a construct encoding a protein into a cell, wherein the encoded protein comprises a cysteine residue substituted at a desired site for tagging, a tail portion, that has a cysteine residue and a cleavage site, at one end of the protein, and a knob portion at the end of the tail portion, providing conditions that allow for the expression of the construct, lysing the cell, and purifying the protein based on the characteristics of the knob on the protein.

In another aspect of the inventions, methods for using the site specifically modified proteins knobs are disclosed. The protein knobs may be used, for example, to map distances between proteins, probe the surface of a protein-protein interface, form a complex between two unrelated proteins, probe the structure and function of the protein knob protein, to immobilize proteins on surfaces, to deliver proteins to cells, as a targeting protein, and for protein purification. Those skilled in the art will recognize that a protein tagged at a specific site with a customized tag is a valuable research tool with a wide range of applications.

### BRIEF DESCRIPTION OF THE DRAWINGS

FIG. 1. Three-dimensional view of hCG. Illustration of the hCG α subunit residues that can be scanned by the hCG β-subunit carboxyterminus. The α- and β-subunit backbones are shown in dark and light gray ribbons, respectively. The β tail is shown as a black ribbon. The locations of the C_{α} carbons of cysteine substitutions that enabled efficient crosslink between the α subunit residue and the probe cysteine are shown as dark spheres. The lighter gray spheres refer to residues that gave less amounts of crosslink. The small pale spheres refer to cysteine substitutions that led to negligible amount of crosslink. Note that α-subunit residues 90, 91, and 92 appear to be too mobile to be seen in the crystal structure of hCG and the arbitrary positions of these residues shown here are intended only to emphasize their apparent abilities to be latched to the seatbelt.

FIG. 2A. Binding of hCG or hCG analogs in which an a subunit residue in loop 2 had been substituted by a cysteine.

FIG. 2B. cAMP accumulation of hCG or hCG analogs in which an a subunit residue in loop 2 had been substituted by a cysteine.

FIG. 3A. Influence of cysteine substitutions in alpha subunit carboxyterminus. Binding of hCG or hCG analogs in which an α subunit residue at the carboxyterminus had been substituted by a cysteine.

FIG. 3B. Influence of cysteine substitutions in alpha subunit carboxyterminus. cAMP accumulation of hCG or hCG analogs in which an α subunit residue at the carboxyterminus had been substituted by a cysteine.

FIG. 4A. Binding of hCG and hCG analogs with β tail attached to the α subunit residues in loop 2.

FIG. 4B. cAMP accumulation of hCG or hCG analogs in which the β tail was attached to the α subunit residues in loop 2.

FIG. 5A. Binding of hCG and hCG analogs with β tail attached to the α subunit residues at the carboxyterminus.

FIG. 5B. cAMP accumulation of hCG or hCG analogs in which the β tail was attached to the α subunit residues in the carboxyterminus.

FIG. 6. Binding and signal transduction activities of the analogs in which BLA was attached to the α subunit residue.

FIG. 7: Amino acid sequences of the α-subunit and mutants having a substituted cysteine. (Note, the mutations are upper case and highlighted. These were prepared by standard cassette mutagenesis and PCR mutagenesis methods that are standard in the art.) (SEQ ID NO: 1-SEQ ID NO: 35).

FIG. 8: Amino acid sequences of the β-subunit analogs. (Note, the cysteine substituted is in uppercase and highlighted.) (SEQ ID NO: 36-SEQ ID NO: 42).

FIG. 9A: Protein Knob with the knob attached at the carboxyterminal end of the protein.

FIG. 9B: Protein Knob with the knob attached at the aminoterminal end of the protein.

FIG. 10A: Protein Knob where the knob is comprised of a cysteine residue.

FIG. 10B: Protein Knob where the knob is comprised of an amino acid sequence including a cysteine residue fused to a protein.

FIG. 10C: Protein Knob where the cysteine residue of the knob is located on the surface of the protein that is the knob.

FIG. 11: Effect of Knob on FSH Activity.

FIG. 12: Summary of Activity of Crosslinked Chimera Analogs in LHR and FSHR Assays Relative to CF101-109, a Bifunctional Chimera with no hCG β-subunit carboxyterminus.

FIG. 13: Effect of Knob on FSH Receptor Signaling.

FIG. 14A: Effect of Knob on LH Receptor Signaling.

FIG. 14B: Effect of Knob on LH Receptor Signaling.

FIG. 15: Amino acid sequences of other analogues (SEQ ID NO: 43-SEQ ID NO: 53).

FIG. 16: Signal transduction and binding activities of LONG and SHORT heterodimers.

FIG. 17: Lutropin activity of hCG Analogs having β-lactamase knobs,

FIG. 18: cAMP accumulation in hCG or Analogs lacking αAsn52 oligosaccharide.

FIG. 19: Binding of αK44A+hCGβ to LHR.

FIG. 20A: Binding of hCG and hCG analog αK44E, K45Q+hCGβ to LHR.

FIG. 20B: LHR cAMP response to hCG and αK44E, K45Q+hCGβ.

FIG. 21A: Binding of hCG and hCG analog αK91E+hCGβ to LHR.

FIG. 21B: Relative activities of hCG and αK91E+hCGβ in LHR cAMP accumulation assays.

FIG. 22A: LHR cAMP response to hCG and αK91M+hCGβ.

FIG. 22B: LHR binding of hCG and αK91M+hCGβ.

FIG. 23: LHR binding of hCG and analogs containing shortened linkers.

FIG. 24: LHR binding of hCG and an analog containing a shortened linker.

FIG. 25: Stimulation of LHR cAMP by hCG and αN52C+hCGβ, S138C.

FIG. 26: LHR binding of analogs in which a truncated hCG β-subunit carboxyterminal tail was used at the carboxyterminal end of the α-subunit to add a knob to hCG β-subunit residues 96, 97, or 99.

FIG. 27: LHR signaling of analogs in which a truncated hCG β-subunit carboxyterminal tail was used at the carboxyterminal end of the α-subunit to add a knob to hCG β-subunit residues 98 or 99.

FIG. 28: LHR binding of analogs in which a truncated hCG β-subunit carboxyterminal tail was used to add a knob to β-subunit residues 95 or 96.

FIG. 29: LHR signaling of analogs in which a truncated hCG β-subunit carboxyterminal tail was used to add a knob to β-subunit residues 95 or 96.

FIG. 30: LHR signaling of analogs in which a GGC tail was used to add a knob to β-subunit residue 96 compared to one lacking a tail and one in which a truncated hCG β-subunit carboxyterminal tail was used to add a knob to β-subunit residue 96 of an hCG/hFSH chimera.

FIG. 31: LHR signaling of analogs in which a truncated hCG β-subunit carboxyterminal tail was used to add a knob to β-subunit residues 98 or 99.

FIG. 31: LHR signaling of analogs showing the influence of the tail on adding a knob to hCG β-subunit residue 95 and on an analog lacking the ability to glycosylate the α-subunit at residue 52.

FIG. 32: Cyclic AMP response to crosslinked analogs.

### DETAILED DESCRIPTION OF THE INVENTION

Currently, most tagged proteins are prepared as fusion proteins with the tag added to the aminoterminal or carboxyterminal end of the protein or with a few residues inserted into a protein loop. While the terminal tags can be of any size, the tags inserted into a protein loop are usually limited to relatively few amino acid residues unless they are inserted between protein domains. Proteins can also be tagged at different sites through the introduction of cysteines at the site to be tagged and then reacting the sulfhydryl group of the cysteine with a sulfhydryl-specific reagent. This may be quite difficult to accomplish, however, when the protein contains other cysteines or when it is prepared under conditions that cause the cysteine to become “blocked” as is often the case when proteins are expressed in eukaryotic cells.

The present invention provides for improved methods for labeling or tagging proteins. The methods of the present invention avoid the complications associated with labeling or tagging by introduction of a cysteine, as described above, and allow various sized probes or knobs to be attached onto the surface of a protein other than at its terminal ends. The present invention provides for methods to add “knobs” of varying size to defined locations on the surface of hCG. The knobs can be as small as a single cysteine residue. The knobs can be a short peptide, such as residues surrounding hCG β-subunit residue 138. The knobs can also be as large as an entire protein. For example, β-lactamase can be used as a knob, The knobs are added during protein synthesis making it unnecessary to remove any blocking residues or attach any protecting groups, tasks that can cause protein denaturation.

The present invention employs a new strategy for attaching a wide range of probes or tags to specific sites on a protein surface based on the finding that a flexible β-subunit tail is capable of being crosslinked to one of several α-subunit residues that have been replaced by a cysteine. Knobs can be a fluorescent protein, such as green fluorescent protein or related molecule. They can have the ability to bind other molecules, properties found in ligands or receptors. They can be proteases, toxins, antibodies or antibody fragments, sequences such as those found in the TAT protein of the human immunodeficiency virus that enable proteins to be translated across membranes, nucleic acids, or oligosaccharides.

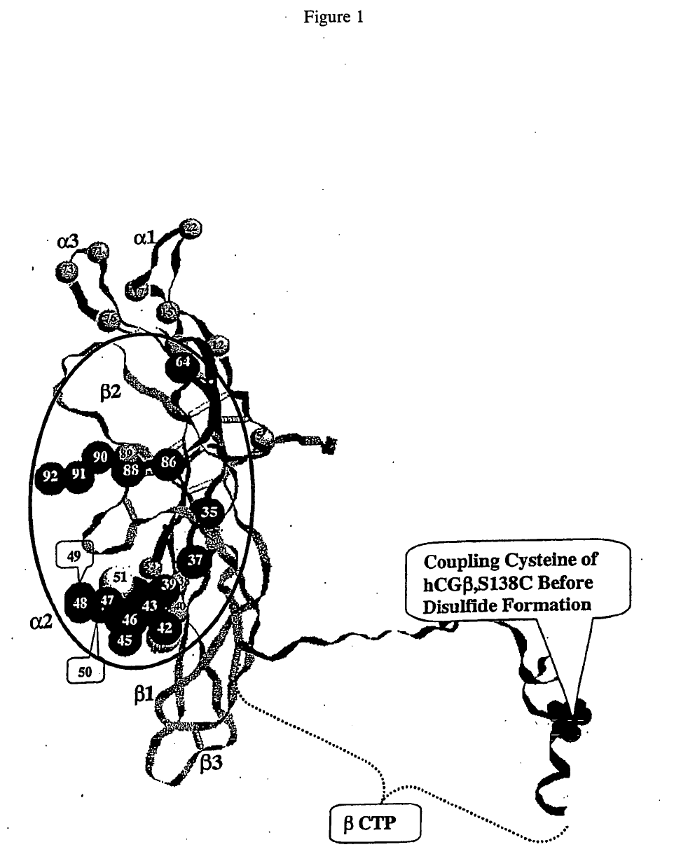

In one embodiment, a composition comprising a protein portion, wherein the protein portion contains a substituted cysteine residue at the desired location to be tagged, a tail portion at the terminal end of the protein portion; and a knob, wherein the knob is located at the free terminal end of the tail portion and contains a cysteine residue, and wherein the cysteine residue of the knob forms a disulfide with the substituted cysteine in the protein portion. The term “protein portion” refers to any protein or polypeptide. The term “tail portion” refers to a stretch of amino acids of a sufficient length to permit the cysteine in the knob to form a disulfide with the substituted cysteine in the protein portion. The tail portion may comprise a native polypeptide portion of the protein of the protein portion, as the β-subunit carboxyterminus does in hCG (see FIG. 1), or may comprise a non-native polypeptide added to a terminal end of the protein portion. The tail portion should also lack residues that will prevent the knob from attaching to the substituted cysteine, such as transmembrane domains or residues that will create a site for binding by other proteins. The term “knob” refers to a cysteine and any residues on either side of the cysteine that are located adjacent to the free terminal end of the tail. The knob may include a single cysteine residue, a linear stretch of amino acids that contains a cysteine, a linear stretch of amino acids fused to a protein, where the cysteine is located in the stretch of amino acids, or a protein that contains a cysteine residue on its surface. A knob can be engineered for a particular purpose or use resulting in a customized tag or probe. For example, the knob may be an epitope tag, a signal sequence, a sequence with high specificity for a bead on a purification column, an enzyme, or a targeting protein.

In another embodiment of the invention, methods for tagging a protein at a specific site are disclosed., The method involves selecting a desired protein, locating a site on the desired protein to be tagged, and selecting a desired knob. The desired knob must contain a cysteine residue. The method further involves preparing a construct encoding the desired protein, a tail portion and the desired knob. The desired protein encoded by the construct includes a cysteine residue substituted at the site to be tagged. The construct is then inserted into a cell for expression of the tagged protein, wherein the cysteine in the knob and the substituted cysteine in the desired protein form a disulfide bond.

The term 37 construct” refers to a nucleic acid vector comprising a promoter linked to an expression cassette engineered to encode a particular protein product. The construct further includes all necessary sequences so that the encoded protein can be expressed and any sequences that may be included to control the expression of the cassette. These sequences may include, but are not limited to, a promoter or initiation sequence, an enhancer sequence, termination sequence, RNA processing signals, and/or a polyadenylation signal sequence. The term “necessary sequences for the expression of the expression cassette” refers to sequences required to ensure the RNA transcripton and subsequent translation of the expression cassette to produce a protein product. The term “promoter” refers to a DNA sequence that is bound by RNA polymerase and is required to initiate RNA transcription of a gene. There are a number of promoters that are known in the art, including those that can enhance or control expression of the gene or expression cassette. The constructs of this invention may be modified by PCR and cassette mutagenesis to create a construct that encodes the desired protein knob.

Knobs at specific sites on a protein can be used to probe the distance of the tested residues to the interface of a receptor or another protein. For example, knobs can be utilized to determine the proximity of the α subunit residues to the receptor binding sites in hCG. Attachment of probes to residues that are located at the binding pocket would abolish the binding activity indicating that the tag is located in the binding pocket. Another advantage of using knobs at specific sites is that larger probes can be used to identify the residues that are close to the protein-receptor interface. This probing strategy can also be used to add epitope tags or signal sequences to any desired site on the protein surface. Additionally, the protein knobs can also be widely used for protein immobilization and protein targeting. If a protease recognition site is engineered within the flexible tail, the tail can then be cleaved after the crosslink is accomplished, leaving the probe tethered by a disulfide to the protein surface, but not tethered by the tail.

Uses for the protein knobs produced using the methods of the invention are not limited merely to inferring the distance between sites on proteins. If a targeted protease were desired, many different proteases could be attached to either the aminoterminus or the carboxyterminus of a protein simply by fusing the coding sequences of the protease to the 5′ or 3′ end of the coding sequence of the protein to be modified as has already been described for the preparation of fusion proteins. (U.S. Pat. No.6,300,099, Sledziewski et al.) Unfortunately, the proximity of the protease to the protein could result in the destruction of the protein.

The use of protein knobs solves this problem because in using the strategy for attaching a knob to a protein as described herein, the protease may be held in a position that makes it incapable of attacking the molecule to which it is attached. Furthermore, the orientation of the protease may enable it to catalyze the cleavage of a desired substrate, such as a receptor. A protease may be engineered onto hCG using the methods described herein such that the protease would cleave the LH receptor preferentially to any other protein, resulting in a loss of lutropin activity. This protease protein knob may be used in a therapeutically desirable fashion for treating polycystic ovary syndrome, a cause of nearly one-third of all human infertility.

In another embodiment of the invention, the compositions and methods of this invention may be used to promote the stable association of two proteins. The data provided herein show that the crosslinked proteins analogs of hCG are much more stable than native hCG at low pH. The introduction of intersubunit disulfides into hCG increases the stability of the heterodimer, (Matzuk, M. M. & Boime, I. (1988) J. Cell Biol. 106, 1049-1059; Heikoop, J. C.; van,den Boogaart; Mulders, J. W.; Grootenhuis, P. D., (1997), Structure-based design and protein engineering of intersubunit disulfide bonds in gonadotropins, Nature Biotechnology 15: 658-662). Previously, intersubunit disulfide bonds have been introduced into the protein based on its crystal structure. The present invention discloses methods for introducing intersubunit disulfides into two proteins during their synthesis when a high resolution structure is not available.

In another embodiment of the invention, the compositions and methods may be used to promote the stable association of DNA polymerase to DNA. The introduction of a linker that wraps around the DNA and is stabilized to the polymerase by a disulfide bond would be expected to stabilize the polymerase to DNA, thereby increasing the length of the resulting transcript.

In yet another embodiment of the invention, the compositions and methods of the invention may be used to produce protein heterodimers of the glycoprotein hormones that lack one or more of their oligosaccharides. Elimination of the glycosylation signal on loop 2 of the α-subunit of hCG reduces the ability of mammalian cells to secrete the heterodimer and to elicit a biological response (Einstein, M., Lin, W., Macdonald, G. J. & Moyle, W. R. (2001) Exp. Biol. Med. 226, 581-590; Slaughter, S., Wang, Y. H., Myers, R. V. & Moyle, W. R. (1995) Mol. Cell. Endocrinol. 112, 21-25; Yen, S. S. C., Llerena, O., Liffle, B. & Pearson, O. H. (1968) J. Clin. Endocrinol. Metab. 28, 1763-1767; Matzuk, M. M.; Boime, I., (1989), Mutagenesis and gene transfer define site-specific roles of the gonadotropin oligosaccharides. Biol. Reprod. 40: 48-53). As shown here, co-expression of hCGβ138C and αN52C, an α-subunit analog in which cysteine is substituted for α-subunit residue Asn52, enabled production of a heterodimer in amounts comparable to hCG. This mutation of the α-subunit eliminated its glycosylation signal and led to an hCG analog that had considerably greater efficacy than an analog of hCG in which the α-subunit loop 2 oligosaccharide had been eliminated by glycanase digestion.

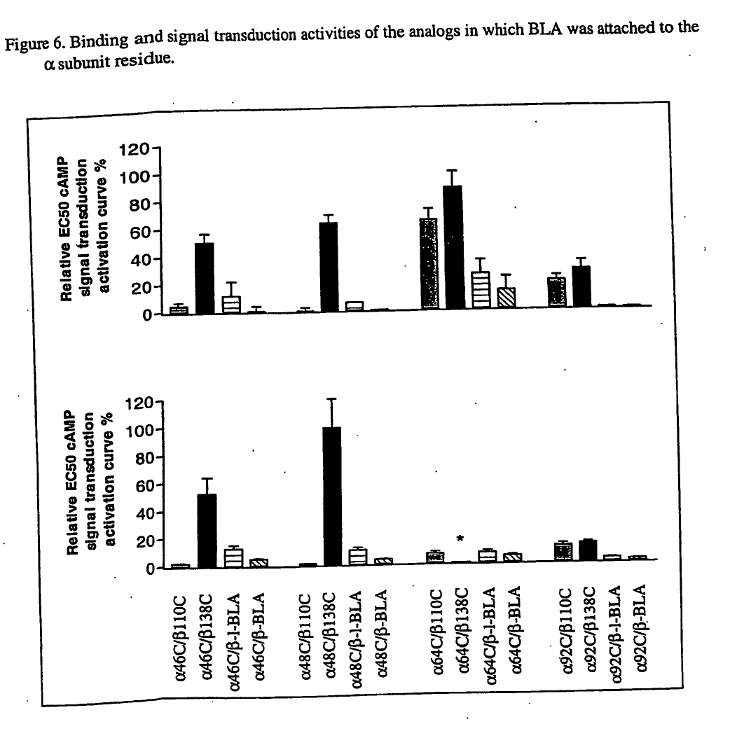

In yet another embodiment, methods for using the compositions and methods of this invention to promote the formation of protein multimers in which the subunits have little or no affinity for one another are disclosed. For example, the data provided in FIG. 6 illustrates that it is possible to attach the enzyme β-lactamase to hCG at one of several different sites. β-lactamase is not known to associate with hCG. By introducing a protease cleavage site, between the hCG β-subunit and the cysteine on the seatbelt, that cleaves the β-subunit carboxyterminus, it would be possible to prepare heterotrimers in which β-lactamase or other proteins are attached stably to nearly any site on hCG.

In another embodiment, the cysteine on the knob may be moved to a site on the surface of the knob portion. This would permit the direct attachment of the knob to the protein in a desired orientation and site on the protein.

In another embodiment, the methods of this invention may be employed to tag proteins with epitope tags. Epitope tags are often attached to proteins to facilitate detection of protein-protein or protein-macromolecule interactions. In the past, epitope tags were attached to the aminoterminal or carboxyterminal end of the protein. However, many epitope tags work only at one end of the protein. Furthermore, the utility of epitope tags added to the ends of proteins are decreased considerably when the ends of a protein are involved in the protein's function. The methods of this invention permit the localization of epitope tags at sites other than the ends of the protein, rendering the epitope tags far more useful. Introduction of a cleavage site into the tail portion would free the end of the protein without disrupting the epitope tag.

In another embodiment of the invention, aldehyde residues may be introduced at specific sites in proteins. Aldehydes are very desirable reactive groups that are not normally found in proteins and can be used to attach several different reagents such as fluorophores to the surface of the protein. This procedure takes advantage of the well known reactivity of aminoterminal serine or threonine residues to gentle periodate oxidation (Yoo, J., Ji, I. & Ji, T. H. (1991) J. Biol. Chem. 266, 17741-17743; Geoghegan, K. F.; Stroh, J. G., (1992) Site-directed conjugation of nonpeptide groups to peptides and proteins via periodate oxidation of a 2-amino alchol. Application to modification at N-terminal serene. Bioconjug. Chem. 3: 138-146). Thus, a serine residue can be introduced immediately after an enzyme cleavage site on a protein. For example, a site recognized by enterokinase can be introduced immediately aminoterminal to a serine, which would be aminoterminal of the cysteine to be used to crosslink the knob to the target cysteine at the site to be tagged. This would result in the sequence Xᵢ-Asp-Asp-Asp-Asp-Lys-Ser-Yₘ-Cys-Zₙ, (SEQ ID NO: 56) where X, Y, and Z refer to any tail portion amino acids and I, m, and n refer to the lengths of the tail portion amino acids. Production of the protein would result in the cysteine in the tail portion becoming crosslinked to the desired site on the protein, which had been changed to cysteine. Cleavage with enterokinase would result in the creation of an aminoterminal serine that is readily oxidized by gentle periodate treatment. The aldehyde that is produced can be readily reacted with any of a variety of hydrazide derived compounds including various fluorophores and biotin. This method would be especially useful in proteins that do not contain unbonded cysteines.

In yet another embodiment, methods for using the compositions of this invention to block specific sites on proteins are disclosed. For example, it would be desirable to block the active site of an enzyme or a toxin that has the potential to kill cells until this activity is desired. Cancer therapy may be an excellent area for use of the compositions and methods of this invention. Thus, a cysteine could be introduced into the enzyme or toxin at a site near the active site of the enzyme or toxin. Addition of a knob that contains a cysteine to the aminoterminal or carboxyterminal end of the protein that has the ability to form a disulfide with the cysteine near the active site would cause the knob to prevent the active site from interacting with its target. The knob may comprise a targeting protein fused to the end of the tail portion which would enable the enzyme/toxin-targeting protein complex to dock with a specific target on the surface of a cell. Treatment of the complex with a protease that cleaves the tail portion would expose the active site of the enzyme or toxin. This strategy could be used to hide the activity of an enzyme or toxin until it reached a site that contained an enzyme that cleaved the tail portion, thereby exposing the toxin. For example, this could occur after internalization of the complex into the cell.

Another use of a knob would be to prevent undesirable associations between proteins that normally complex with one another. Thus, the disulfide would be designed to hold the knob in a position on the interface between the two proteins.

In yet another embodiment, the present invention provides a method for precise separation of a target protein with only a few steps. Employing the methods described herein, an expression construct encoding the protein of interest, a tail portion and a knob is created. The location of the target cysteine residue on the protein of interest may be at the carboxyterminal end, the aminoterminal end, or at any desired location on the surface of the protein of interest. Once the coupling cysteine on the knob binds to the cysteine on the desired protein after construct expression, the resulting protein-knob complex is run through a column. With proper choice of a tightly-binding knob, the protein-knob complex binds to the affinity resin and the unbound proteins and cellular components are washed off. Then, the complex is eluted, the knob is cleaved off, and only the purified protein remains. For example, (Strategene's Affinity™) pCAL vector may be used and the calmodulin-binding peptide (CBP) may be chosen as the knob. The CBP-knob binds to the calmodulin resin and can be eluted with 2 mM EDTA at a neutral pH, thus avoiding harsh elution conditions that may denature proteins. However, there are many possible vector and knob combinations that may be utilized with the present invention.

In a further aspect of the invention related to protein purification, the protein of interest may be constructed so that the protein knob has a short tail with a cleavage site. The knob tail must be short enough so that the knob cannot form a disulfide linkage with the protein itself. Instead, the structure of the protein and tail are such that the knob is conducive for forming a disulfide bond with another protein. In solution, the proteins with short tails would line up like a string of beads, connected by the disulfide bond between the cysteine on a first protein's knob and the cysteine on the protein portion of a second protein. The knob of the second protein would then form a disulfide bond with a cysteine on the protein portion of a third protein and so on. The resulting chain of proteins may be run on a sucrose gradient and centrifuged, the chain would fall to the bottom of the gradient due to its weight. Finally, to separate the protein chains from other heavy material such as lysed cell membranes, the bottom layer where the chains reside would be treated with an enzyme specific for the cleavage site in the short tail, resulting in individual proteins. The mixture would then be recentrifuged. The individual proteins would stay near the top of the gradient, allowing for easy removal and purification. This chain of proteins could also be purified using other methods discussed above or other methods known in the art for protein purification.

In another aspect of this invention, the protein of interest may be constructed so that each protein knob interacts with other protein knobs to form a grid-like structure. The tails should be constructed so that the knob is unable to react with the cysteine on the protein portion, similar to the tails in the string of beads method. The proteins may also comprise tails and knobs at both terminal ends of the protein. The protein portion should comprise multiple substitute cysteine residues so that knobs from more than one separate protein may form disulfide bonds with the protein. The number and location of substituted cysteines on the protein knobs would have to be determined in advance of construction using a computer modeling program or other method. In solution, the proteins would form a grid-like structure as a single protein may form disulfide bonds with more than one protein. This matrix of protein knobs could then be purified by any of the above methods, keeping in mind that the high molecular weight of the structure would make it a good candidate for centrifugation techniques.

In another embodiment, the present invention may be used to add cysteines to proteins. It is often desirable to introduce cysteines into proteins to take advantage of the unique reactivity of cysteine for attaching the proteins to surfaces and for attaching other molecules such as fluorophores to the protein containing the additional cysteine. Many proteins contain cysteines or disulfides, making it difficult to utilize the cysteine that had been introduced into the molecule. By introducing a tryptophan into the tail adjacent to the cysteine that is used to crosslink the knob to the cysteine that had been introduced at the site of the protein to be modified, this difficulty can be circumvented. Due to differences in the absorption spectra of tryptophan, tyrosine, and phenylalanine, irridation of the protein with light at 295 nm will selectively target the tryptophan residues. This will cause the disruption of the adjacent disulfide, making the thiol residue on the desired cysteine reactive. Irridation in the presence of the group that is to be added to the protein will enabled the protein to become labeled at the desired site, even in proteins that contain several other disulfides. While it is conceivable that other disulfides near tryptophan residues will also be made reactive, the fact that most proteins contain few tryptophans means that this is usually not a problem.

In yet another embodiment, the present invention may be used to create and obtain high yields of heterodimeric proteins. When creating an expression construct for the protein knob one dimer of the heterodimer may comprise the protein portion. If the dimer of the protein portion does not have a naturally occurring tail portion, a tail portion may be fused to the dimer of the protein portion. The knob may comprise the other dimer of the heterodimer. Upon expression of the construct, there is a higher likelihood that the. heterodimer will form due to the tail portion that connects the dimers and the capability of forming the disulfide bond. This method avoids problems of homodimer formation where dimers of a heterodimer are co-expressed in the same cell.

### EXAMPLE 1

### Protein Knobs to Study hCG-LHR Interaction

The sources of hCG and antibodies used in these studies have been described (Bernard, M. P., Myers, R. V. & Moyle, W. R. (1998) Biochem. J. 335, 611-617; Moyle, W. R., Campbell, R. K., Rao, S. N. V., Ayad, N. G., Bernard, M. P., Han, Y. & Wang, Y (1995) J. Biol. Chem. 270, 20020-20031; Moyle, W. R., Matzuk, M. M., Campbell, R. K, Cogliani, E., Dean Emig, D. M., Krichevsky, A., Barnett, R. W. & Boime, I. (1990) J. Biol. Chem. 265, 8511-8518.) A construct capable of expressing hCGβ-S138C was prepared by cassette mutagenesis between the natural Apal site in the hCG cDNA and a BamHI site that had been engineered downstream of the termination codon as described (Campbell, R. K., Dean Emig, D. M. & Moyle, W. R. (1991) Proc. Natl. Acad. Sci. (U.S.A.) 88, 760-764.; Campbell, R. K., Dean Emig, D. M. & Moyle, W. R. (1991) Proc. Natl. Acad. Sci. (U.S.A.) 88, 760-764.) Vectors used to express α-subunit cysteine substitutions were also prepared as described (Xing, Y., Lin, W., Jiang, M., Myers, R. V., Cao, D., Bemard, M. P. & Moyle, W. R. Alternatively folded choriogonadotropin analogs: implications for hormone folding and biological activity. Journal of Biological Chemistry. 2001.) Constructs encoding the human α-subunit or cysteine substituted analogs were co-expressed with the hCG β-subunit or hCGβ-S138C in COS-7 cells as described (Campbell, R. K., Dean Emig, D. M. & Moyle, W. R. (1991) Proc. Natl. Acad. Sci. (U.S.A.) 88, 760-764.) Materials secreted into the culture media were assayed by sandwich immunoassays (Moyle, W. R., Ehrlich, P. H. & Canfield, R. E. (1982) Proc. Natl. Acad. Sci. (U.S.A.) 79, 2245-2249) employing α-subunit antibody A113 for capture and radioiodinated β-subunit antibody B110 for detection. They were treated at acid pH to promote the dissociation of heterodimers that lack a disulfide crosslink, also as described (Xing, Y., Lin, W, Jiang, M., Myers, R. V., Cao, D., Bernard, M. P. & Moyle, W. R. Alternatively folded choriogonadotropin analogs: implications for hormone folding and biological activity. Journal of Biological Chemistry. 2001.) Chinese hamster cells (CHO cells) that overexpress the rat LHR were used to monitor the influence of the analogs on the ability of ¹²⁵I-hCG to bind LHR binding and elicit cyclic AMP accumulation as reported previously (Bemard, M. P., Myers, R. V. & Moyle, W. R. (1998) Biochem. J. 335, 611-617; Moyle, W. R., Matzuk, M. M., Campbell, R. K, Cogliani, E., Dean Emig, D. M., Krichevsky, A., Barnett, R. W. & Boime, I. (1990) J. Biol. Chem. 265, 8511-8518; Moyle, W. R., Campbell, R. K, Myers, R. V., Bernard, M. P., Han, Y. & Wang, X. (1994) Nature 368, 251-255.)

An alternative approach to deciphering hCG-LHR interactions involves identifying surfaces of the hormone that remain exposed in the hormone-receptor complex (Moyle, W. R., Matzuk, M. M., Campbell, R. K, Coglieni, E., Dean Emig, D. M., Krichevsky, A., Barnett, R. W. & Boime, I. (1990) J. Biol. Chem. 265, 8511-8518.) Through a process of elimination, areas that remain exposed in the hormone-receptor complex are then mapped on the surface of the hormone to reveal sites capable of contacting the receptor. Since these data are collected during studies in which the hormone retains its ability to interact with receptors, the data are more readily interpreted than those dependent on changes in hormone receptor interactions. Most methods to detect non-contact residues have relied on the use of monoclonal antibody probes, a procedure that is severely limited in resolution. To circumvent this limitation, the activities of analogs in which the seatbelt was latched to the α-subunit were measured

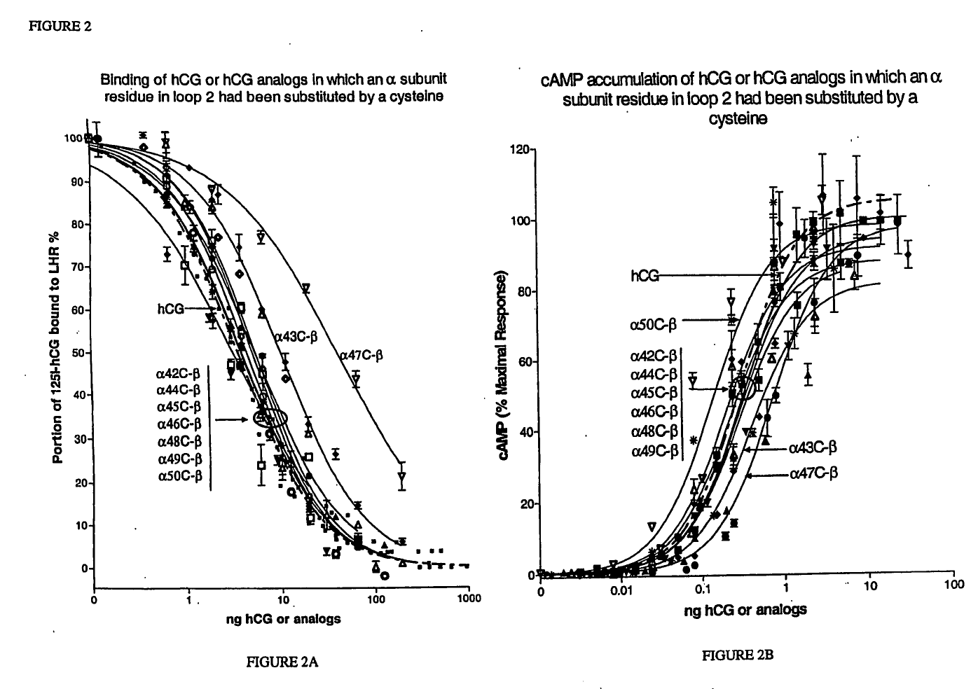

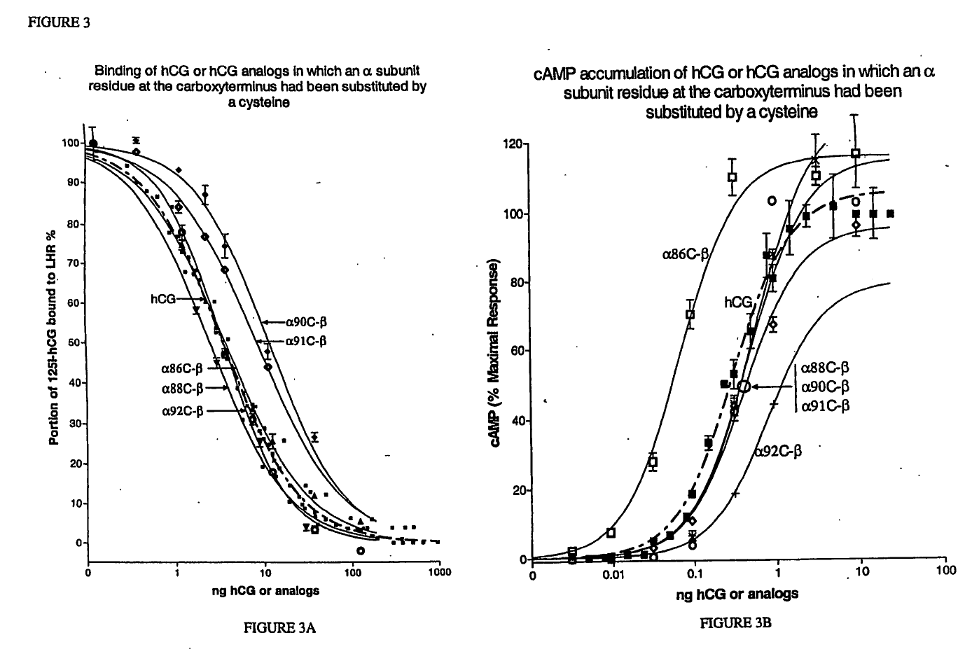

(Xing, Y., Lin, W., Jiang, M., Myers, R. V., Cao, D., Bemard, M. P. & Moyle, W. R. Alternatively folded chotiogonadotropin analogs: implications for hormone folding and biological activity. Journal of Biological Chemistry. 2001.) Several of these analogs had essentially the same activity as hCG, even though they contained parts of the seatbelt and the β-subunit carboxyterminus attached to areas that might be expected to block hormone receptor interaction. Linking the seatbelt to the α-subunit has the potential to alter the conformation of the heterodimer; a phenomenon that may have been responsible for the low activity of some analogs. Recently, it was found that the long disordered carboxyterminal end of the β-subunit was sufficiently mobile to scan much of the surface of the heterodimer until a cysteine introduced at residue 138 formed a disulfide with the cysteine introduced into the α-subunit. The seatbelts of these analogs are latched as they are in hCG, making it less likely that the mutation alters the conformation of the heterodimer. As described here, many of these hCG analogs are much more active than those in which the seatbelt had been attached to the β-subunit. The results of the studies performed also show that much of hCG α-subunit loop 2, a portion of the hormone likely to be near the receptor interface, does not contact the LHR. The activities of hCG analogs before and after a portion of the disordered β-subunit carboxyterminus had been tethered to cysteines introduced into loop 2 and the carboxyterminus of the α-subunit by a disulfide were compared (See FIGS. 2 and 3). Except for analogs with cysteines at the tip of loop 2, heterodimers lacking the crosslink had at least 25% the activity of hCG in LHR binding and signaling assays. This suggests that few residues in either region contribute more than a fraction of the total hCG-LHR binding energy and conflicts with reports that carboxyterminal residues are essential for efficacy. Tethering the β-subunit carboxyterminus probe to the surface of α-subunit loop 2 facing β-subunit loops 1 and 3 had relatively little influence on binding or signaling, indicating this portion of the hormone is unlikely to contact the receptor. The loss of activity caused by linking the β-subunit carboxyterminus to other residues may indicate these portions of the α-subunit are near the receptor. Application of this procedure to the study of hFSH interactions with the FSH receptor revealed that hFSH binds to the FSH receptor in a similar overall fashion but that different parts of hFSH make the key receptor contacts. Application of this procedure to the study of a chimeric hCG-hFSH ligand that has the ability to interact with both LH and FSH receptors confirmed these observations. Thus, different portions of the chimera were found to be closer to the LHR than to the FSHR. Similar mutagenesis strategies should be useful for identifying the surfaces of other proteins that do not participate in protein-protein contacts.

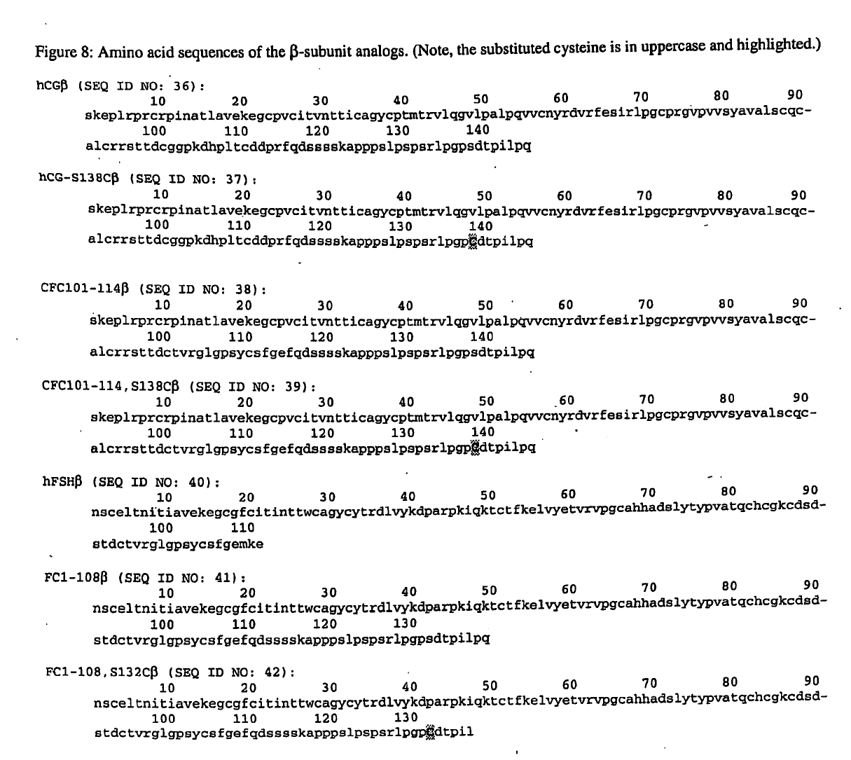

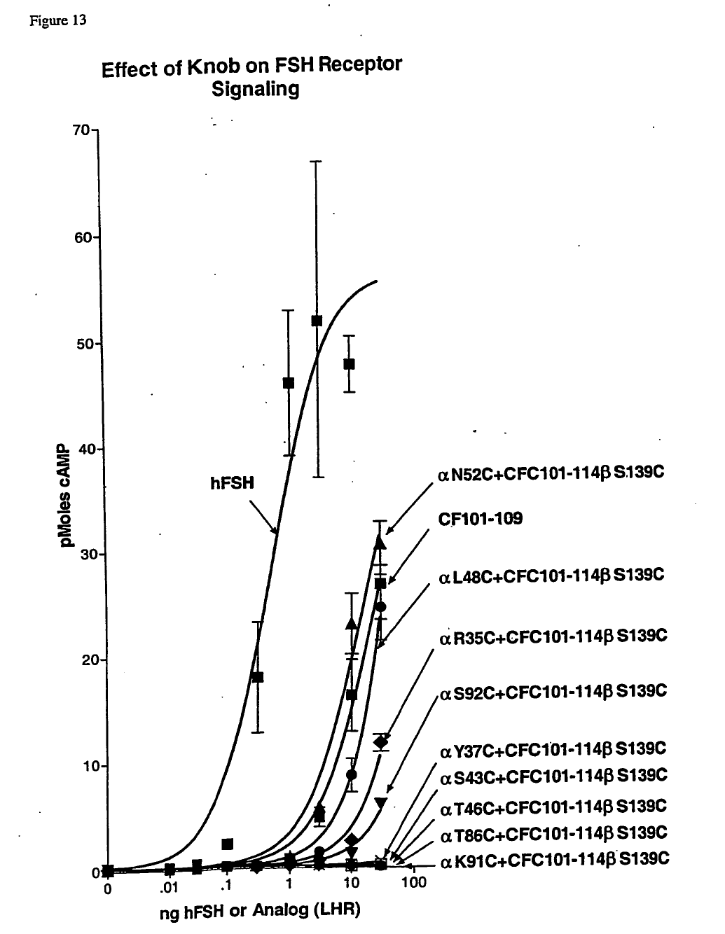

Studies described here also illustrate application of this procedure to identify the binding site of hFSH to the FSH receptor and to compare the interactions of a bifunctional hCG/hFSH analog with both LH and FSH receptors. An analog of hFSH β-subunit that encoded a portion of the residues of the hCG β-subunit carboxyterminus (fqdsssskapppslpspsrlpgpstdpilpg, SEQ ID NO: 55) at its β-subunit carboxyterminus was prepared since hFSH does not have a “seatbelt” (tail portion) (FIG. 8, SEQ ID NO: 41). Similar to the studies performed with hCG in which serine residue 138 was replaced with cysteine, the serine residue 132 of the hFSH β-subunit analog was replaced with a cysteine (FIG. 8, SEQ ID NO: 42). The hFSH β-subunit S132C analog was then expressed with several α-subunit analogs that contained a substituted cysteine. As expected based on the experience with similar hCG analogs, the β-subunit of the hFSH analog became crosslinked with the α-subunit by a disulfide as evidenced by the fact that the heterodimer was much more stable at low pH than was native hFSH. Many of these analogs had high activities in hFSH receptor assays (FIG. 13). Several differences were detected in the activities of the FSH-derived analogs compared with those of the hCG-derived analogs. These differences revealed that FSH interacted with the FSH receptor differently than hCG interacted with the LH receptor thereby enabling the construction of models of each hormone-receptor complex. The results also indicate that the hCG β-subunit carboxyterminus sequence can be used as the ‘tail portion’ for adding a knob to the surface of a protein that lacks a suitable site or ‘tail portion’ for doing so. One skilled in the art will recognize that many sequences other than the hCG β-subunit carboxyterminus could be used to accomplish the same task. A ‘tail portion’ sequence requires both that the sequence be sufficiently long such that the cysteine in the tail portion can reach the cysteine on the surface of the protein to which it is to be attached, and that the sequence not contain residues that prevent it from reaching the cysteine on the surface of the protein. Residues that would prevent the cysteine on the tail portion from reaching the cysteine on the surface of the protein include residues that would cause the sequence to fold into a separate domain that would sequester the cysteine on the tail portion, residues such as those that contain a signal for the protein to become attached to a cellular membrane thereby sequestering the cysteine, residues that contain a signal for the protein to bind another protein thereby sequestering the cysteine, or residues that are highly charged that block interactions between the surface of the protein.

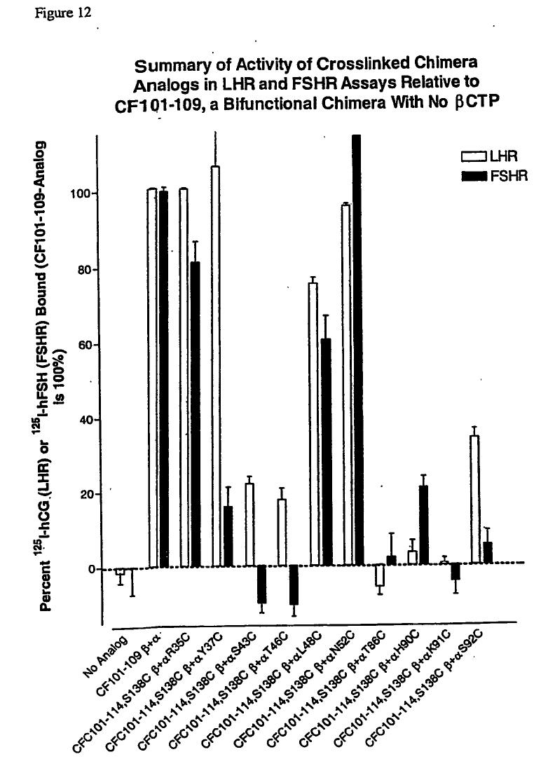

Bifunctional analogs of the glycoprotein hormones can be prepared by interchanging parts of their seatbelts. (U.S. Pat. No. 5,508,261, Moyle et al). To further distinguish differences in the interactions of lutropins, such as hCG with the LH receptor, and follitropins, such as hFSH with the FSH receptor, an analog of hCG that is known to bind both LH and FSH receptors was prepared. The hCG β-subunit residues 101-114 were replaced with their hFSH β-subunit counterarts, namely hFSH β-subunit residues 95-108 (FIG. 8, SEQ ID NO: 38). As done earlier in the case of the hCG and hFSH analogs, serine residue 38 in the β-subunit carboxyterminus of this analog was replaced with cysteine (FIG. 8, SEQ ID NO: 39). The construct was expressed in COS-7 cells with several α-subunit analogs that contained a substituted cysteine. The heterodimers that were produced in COS-7 cells were quantified using a sandwich immunoassay employing antibodies to the α- and β-subunits of hCG. Many were found to be crosslinked by a disulfide between the α- and β-subunits on the basis of their increased stability at low pH. Some of these analogs had significantly different interactions with LH and FSH receptors. For example, the analog in which the β-subunit carboxyterminus was crosslinked to α-subunit residue cysteine 37 interacted well with LH receptors and poorly with FSH receptors (FIGS. 12-14). This confirmed the finding that the interactions of hCG with LH receptors differed significantly from those of hFSH with FSH receptors and provided considerable further support for the models by which each of these hormones interact with their receptors.

Results

hCG α-Subunit Analogs and Either Native hCG β-Subunit or HCGβ-S138C

COS-7 cells that were co-transfected with vectors encoding most of the α-subunit analogs and either the native β-subunit (Table 1) or hCGβ-S138C (Table 2) were capable of assembling the heterodimer and secreting it into the culture medium. Heterodimers secreted poorly or not at all included those containing the β-subunit and α-subunit analogs in which a cysteine had been substituted for residues Tyr37, Pro40, Asn52, and Y89C (Table 1). The poor secretion of αN52C/β may reflect the absence of the N-linked glycosylation signal normally found at this position of hCG that is required for efficient secretion of the heterodimer (Matzuk, M. M. & Boime, I. (1988) J. Cell Biol. 106, 1049-1059.)

TABLE 1
Production of heterodimers by COS-7 cells
transfected with the indicated subunit constructs

| | Amount of dimer in the medium |
|---|---|
| transfection | (ng/50 μl) mean ± SEM |
| αG22C + β | 9.034 ± 0.414 |
| αY37C + β | 0.464 ± 0.052 |
| αP38C + β | 1.710 ± 0.026 |
| αP40C + β | 0.588 ± 0.093 |
| αL41C + β | 7.617 ± 0.438 |
| αR42C + β | 0.962 ± 0.135 |
| αS43C + β | 3.571 ± 0.156 |
| αK44C + β | 2.788 ± 0.358 |
| αK45C + β | 1.090 ± 0.093 |
| αT46C + β | 7.044 ± 0.229 |
| αM47C + β | 9.491 ± 0.524 |
| αL48C + β | 1.497 ± 0.170 |
| αV49C + β | 2.579 ± 0.297 |
| αQ50C + β | 1.985 ± 0.153 |
| αN52C + β | 0.615 ± 0.081 |
| αV53C + β | 6.108 ± 0.356 |
| αM71C + β | 6.153 ± 0.332 |
| αG73C + β | 2.849 ± 0.194 |
| αT86C + β | 4.973 ± 0.027 |
| αY88C + β | 7.930 ± 0.290 |
| αY89C + β | Not Detected |
| αH90C + β | 0.971 ± 0.170 |
| αK91C + β | 1.049 ± 0.055 |
| αS92C + β | 2.595 ± 0.106 |

Most of the α-subunit analogs tested were detected in heterodimers that contained hCGβ-S138C (Table 2). HCGβ -S138C rescued the formation of some analogs that were secreted poorly when expressed with the native β-subunit, including Tyr37, Pro40, and Asn52 but not Tyr89 (Table 2, FIG. 1). Many of these heterodimers in Table 2 appeared to contain an intersubunit crosslink because they were readily detected following a brief treatment at low pH. Heterodimers containing the native α-subunit or the αG22C and αV53C α-subunit analogs were destroyed at low pH suggesting that they lacked an intersubunit disulfide. Only a fraction of the heterodimers that contained the αQ5C, αQ27C, αP40C, αK51C, αL41C, αM71C, and αV76C α-subunit analogs appeared to be stabilized by intersubunit disulfides, however (Table 2). The cysteines in the α-subunit analogs of analogs that formed little or no crosslinked heterodimers are at the subunit interface or are far from β-subunit residue Asp111, the first residue in the carboxyterminal extension on the β-subunit. Consequently, the cysteine at β-subunit residue 138 appears to have been prevented from reaching these α-subunit cysteines. This phenomenon suggests that most intersubunit disulfide crosslinks formed after the subunits had assembled into a heterodimer similar in structure to hCG. The only α-subunit analog tested that failed to form a heterodimer with either the hCG β-subunit or hCGβ-S138C contained a cysteine in place of Tyr89. While this tyrosine is not essential for folding of the α-subunit because it can be deleted or changed to residues other than cysteine without disrupting heterodimer formation (Pierce, J. G. & Parsons, T. F. (1981) Annu. Rev. Biochem. 50, 465-495), replacing the tyrosine with a cysteine may have disrupted folding of the α-subunit (Chen, F., Wang, Y. & Puett, D. (1992) Mol. Endocrinol. 6, 914-919.)

the indicated α-subunit constructs and HCGβ-S138C.

| α-Subunit | Heterodimer | Crosslinked | Seatbelt Latch |
|---|---|---|---|
| Analog | (ng/50 μl) | (%)^{a} | (B111/B110)^{b} |
| Native α | 26.2 ± 0.8 | −6.5 ± 2.6 | Not Done |
| αQ5C | 14.3 ± 1.3 | 17.5 ± 9.9 | 0.98 |
| αG22C | 9.1 ± 0.4 | −3.1 ± 1.0 | Not Done |
| αQ27C | 3.3 ± 0.0 | 21.1 ± 4.2 | 1.03 |
| αR35C | 6.0 ± 0.0 | 98.4 ± 4.9 | 0.97 |
| αY37C | 5.4 ± 1.2 | 80.2 ± 0.5 | 0.18 |
| αP38C | 0.3 ± 0.04 | Not Tested | Not Done |
| αP40C | 2.1 ± 0.04 | 52.8 ± 4.3 | 0.64 |
| αL41C | 3.2 ± 0.04 | 37.2 ± 0.6 | 0.49 |
| αR42C | 22.1 ± 5.3 | 67.7 ± 5.3 | 0.48 |
| αS43C | 29.7 ± 1.4 | 81.3 ± 1.9 | 0.60 |
| αK44C | 4.0 ± 0.1 | 55.4 ± 1.3 | 0.84 |
| αK45C | 5.8 ± 0.1 | 82.9 ± 6.2 | 0.85 |
| αT46C | 21.4 ± 0.4 | 76.8 ± 0.6 | 1.14 |
| αM47C | 4.9 ± 0.8 | 72.0 ± 2.0 | 0.82 |
| αL48C | 14.3 ± 0.9 | 88.5 ± 2.1 | 0.86 |
| αV49C | 6.0 ± 0.5 | 70.5 ± 6.1 | 0.76 |
| αQ50C | 0.8 ± 0.1 | 66.7 ± 7.4 | 0.82 |
| αK51C | 4.1 ± 1.2 | 27.5 ± 1.4 | 0.86 |
| αN52C | 6.3 ± 0.2 | 70.6 ± 3.9 | 0.88 |
| αV53C | 6.1 ± 0.4 | 4.3 ± 2.9 | 1.01 |
| αS64C | 4.4 ± 0.3 | 82.5 ± 8.9 | 0.85 |
| αM71C | 6.2 ± 0.3 | 14.1 ± 3.2 | Not Done |
| αV76C | 4.3 ± 0.0 | 41.9 ± 4.9 | 1.10 |
| αT86C | 7.6 ± 0.8 | 88.4 ± 9.3 | 0.66 |
| αY88C | 7.4 ± 0.3 | 83.0 ± 6.2 | Not Done |
| αY89C | Undetectable | Not Tested | Not Done |
| αH90C | 7.2 ± 1.3 | 79.2 ± 1.0 | 0.86 |
| αK91C | 9.9 ± 5.7 | 69.4 ± 3.3 | 0.91 |
| αS92C | 15.1 ± 0.8 | 140.7 ± 14.4 | 0.98 |

^{a}This value was calculated as the percentage of material that remained in the sample following treatment at acid pH as described in the text.
^{b}This value was calculated as the ratio of activity determined in sandwich assays employing A113 and ¹²⁵I-B111 relative to that observed in A113 and ¹²⁵I-B110 after low pH treatment. Detection of any B111 binding indicates that the seatbelt is latched. The low values observed in some cases may reflect a steric influence of the β-subunit carboxyterminus on the ability of B111 to interact with the crosslinked heterodimer.

Substitution of cysteine for many α-subunit residues in loop 2 had only a small influence on the receptor-binding and signal-transduction activities of hCG (Table 3, FIG. 2). Replacing α-subunit residues αMet47 (Table 3, FIG. 2) and αLys51 (Einstein, M., Lin, W., Macdonald, G. J. & Moyle, W. R. (2001) Exp. Biol. Med. 226, 581-590.) with cysteine reduced the activity of the heterodimer in binding and signaling assays relative to that of hCG. While an analog in which αLys51 had been replaced by alanine also had considerably less activity than hCG (Einstein, M., Lin, W, Macdonald, G. J. & Moyle, W. R. (2001) Exp. Biol. Med. 226, 581-590), hCG-αM47A, an analog in which αMet47 had been replaced by alanine was nearly as active as hCG in both assays (Table 3). This suggested that the presence of a methionine at α-subunit residue 47 was not essential for hCG activity. The specific role of αLys51 in receptor interaction remains to be determined. Based on the finding that a heterodimer in which α-subunit residue 51 is crosslinked to β-subunit residue 99 by a disulfide is more active than those in which αLys51 is replaced by cysteine or alanine, it appears likely that replacing the αLys51 sidechain may alter the conformation of the heterodimer.

TABLE 3
Influence of the mutations in α-subunit loop 2 and carboxyterminus
on heterodimer lutropin activity

| | | | Potency of Acid | |
|---|---|---|---|---|
| | Potencies of Native β subunit Analogs | | Stable hCGβ-S138C Analogs | |
| α-Subunit | (Percent hCG, 95% CL) | | (Percent hCG, 95% CL) | |
| Analog | Receptor-Binding^{a} | Signal Transduction^{a} | Receptor-Binding^{a} | Signal transduction^{a} |
| αR42C | 113 (97-132) | 103 (85-124) | 327 (305-351) | 332 (287-385) |
| αS43C | 35 (31-39) | 74 (50-110) | 17 (15-20) | 43 (37-49) |
| αK44C | 66 (60-72) | 97 (75-124) | 250 (228-274) | 191 (148-247) |
| αK44A | 84 (71-101) | | Not Transfected | Not Transfected |
| αK44E, K45Q | 58 (54-61) | 42 (32-55) | Not Transfected | Not Transfected |
| αK45C | 93 (87-100) | 91 (71-117) | 223 (212-257) | 168 (129-217) |
| αT46C | 38 (35-42) | 56 (43-73) | 50 (44-57) | 52 (43-64) |
| αM47C | 8.7 (7.4-10) | 23 (16-32) | 4.1 (2.9-5.9) | 2.4 (2.1-2.7) |
| αM47A | 83 (71-97) | | Not Transfected | Not Transfected |
| αL48C | 60 (54-68) | 147 (96-226) | 66 (60-72) | 99 (81-120) |
| αV49C | 70 (64-77) | 133 (102-174) | 71 (65-76) | 99 (84-119) |
| αQ50C | 81 (76-85) | 216 (163-285) | | 308 (241-395) |
| αK51C | Undetectable* | Undetectable* | | |
| αN52C | Not Done | Not Done | 61 (57-65) | |
| αV53C | 149 (137-162) | | Not Done | Not Done |
| αS64C | Not Done | Not Done | 89 (78-101) | |
| αT86C | 95 (84-106) | 444 (354-555) | 2.2 (indeterminate) | 0.18 (0.16-0.21) |
| αY88C | 141 (128-156) | 74 (64-84) | 0.05 (indeterminate) | 0.12 (0.09-0.16) |
| αY89C | Not Done | Not Done | Not Done | Not Done |
| αH90C | 30 (26-34) | 65 (48-89) | 3.1 (1.8-5.4) | 1.0 (0.96-1.1) |
| αK91C | 41 (38-45) | 56 (46-68) | 2.2 (1.1-4.4) | Undetectable |
| αK91E | 4.3 (3.4-5.5) | 1.1 (0.1-2.2) | Not Transfected | Not Transfected |
| αK91M | 68 (36-144) | 66 (45-140) | Not Transfected | Not Transfected |
| αS92C | 100 (91-110) | 38 (31-47) | 28 (23-34) | 13 (12-14) |

^{a}Based on the concentration of analog determined by sandwich immunoassay. These values were calculated from the IC50 values from experiments summarized. Several analogs were not tested in these assays due to the fact that only small amounts were produced by transfected COS-7 cells. As also shown in Table 1, we did not obtain any stable heterodimer following acid treatment of α/hCGβ-S138C.

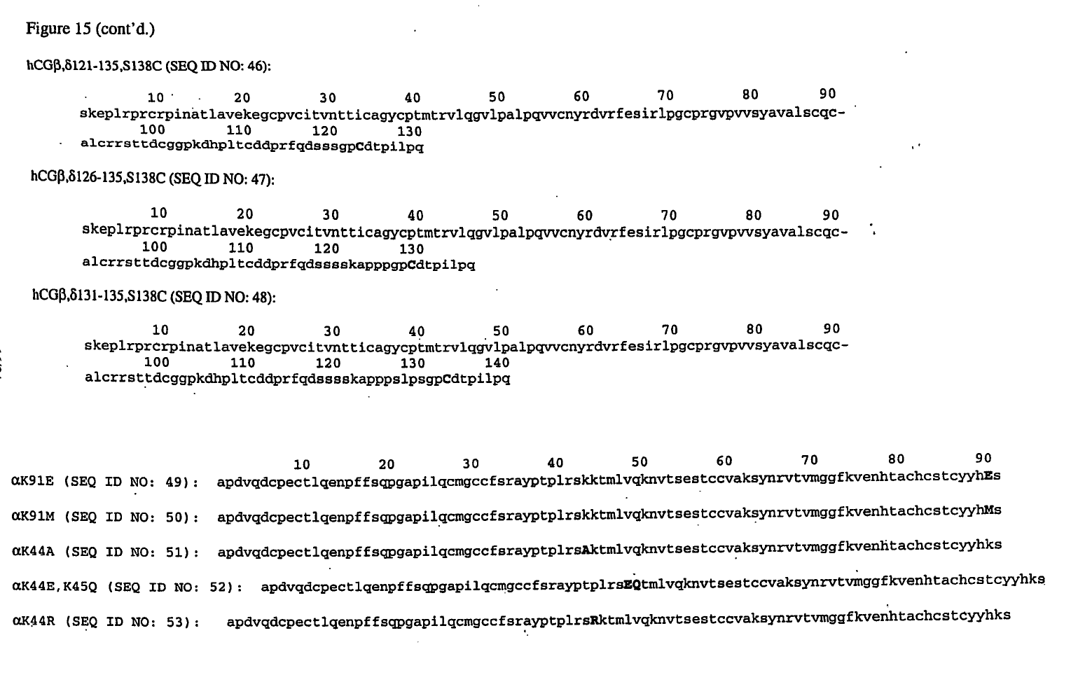

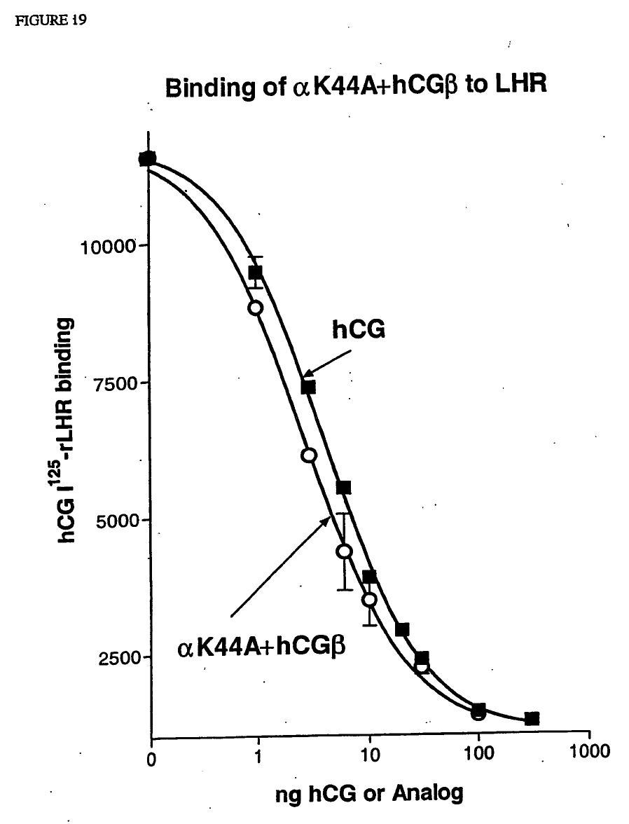

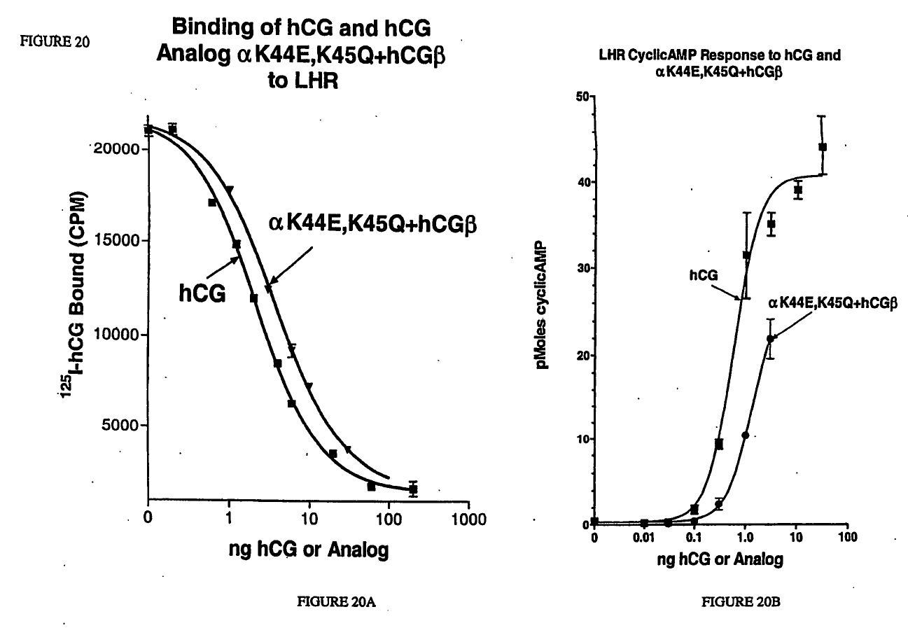

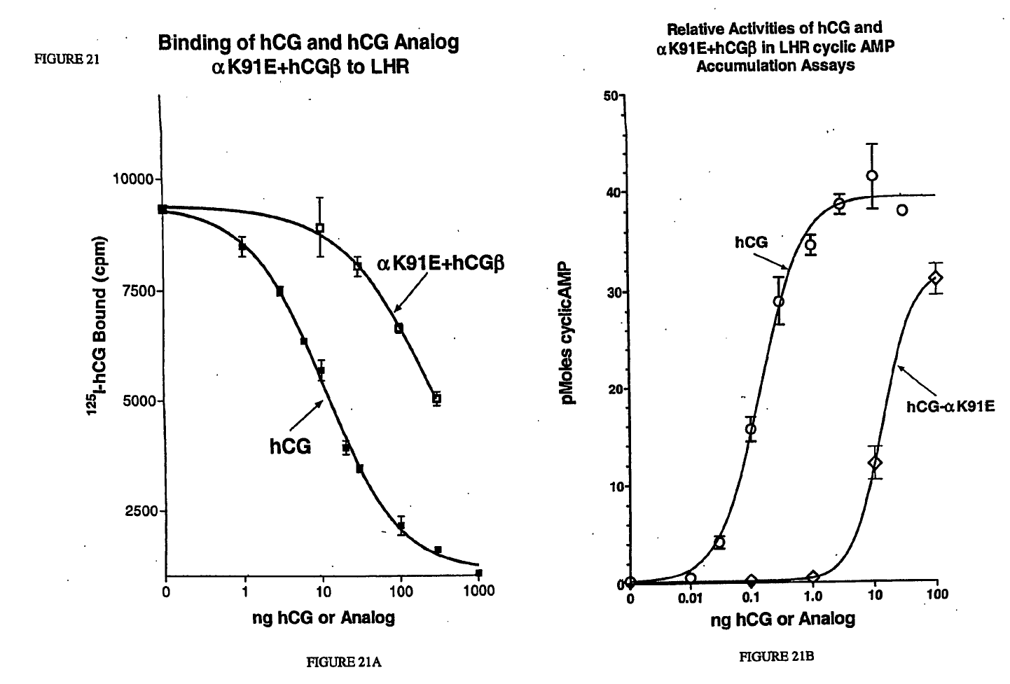

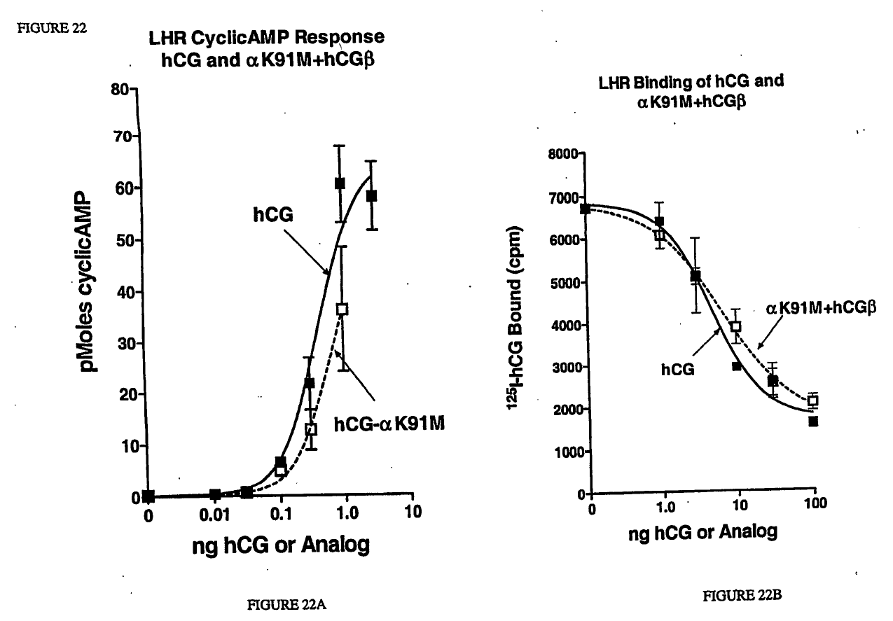

Changing α-subunit loop 2 residue αLys44 to alanine has been reported to reduce hCG activity 100-fold or more (Xia, H., Chen, F. & Puett, D. (1994) Endocrinol. 134, 1768-1770.) Thus, the finding that replacing αLys44 and several nearby α-subunit residues with cysteine had much less influence on hCG activity than expected (Table 3). During part of an unrelated study to test predictions made about the charge on the tip of α-subunit loop 2 on subunit combination (Slaughter, S., Wang, Y. H., Myers, R. V. & Moyle, W. R. (1995) Mol. Cell. Endocrinol. 112, 21-25), an hCG analog that contained glutamate and glutamine in place of the lysines at residues 44 and 45, respectively, was prepared (hCG-αK44E, K45Q) (FIG. 15, SEQ ID NO: 52). Unexpectedly, this analog had high activity in both assays as did hCG-αK44A (FIG. 15, SEQ ID NO: 51) and hCG-αK44R (FIG. 15, SEQ ID NO: 53), analogs that had alanine and arginine in place of αLys44 (Table 3, FIG. 19). The high activity of the latter analog was expected based on the fact that the equine α-subunit has this same substitution (Pierce, J. G. & Parsons, T. F. (1981) Annu. Rev. Biochem. 50, 465-495; Pierce, J. G. & Parsons, T. F. (1981) Annu. Rev. Biochem. 50, 465-495.) These studies were consistent with observations made by replacing these residues with cysteine. These observations were contrary to earlier findings (Xia, H., Chen, F. & Puett, D. (1994) Endocrinol. 134, 1768-1770), suggesting that neither of the highly conserved positively charged lysine residues in the small helix found in α-subunit loop 2 are essential for LHR interactions.

Elimination of βCys26

It was found that βCys110, the cysteine that latches the carboxyterminal end of the seatbelt to the β-subunit core, can become crosslinked to the cysteine that was introduced into many of these α-subunit analogs if the βCys110 is prevented from forming a disulfide with β-subunit residue 26 (Xing, Y., Lin, W., Jiang, M., Myers, R. V., Cao, D., Bemard, M. P. & Moyle, W. R. Alternatively folded choriogonadotropin analogs: implications for hormone folding and biological activity. Journal of Biological Chemistry 50, 46953-46960, 2001.) This finding changed the position of the seatbelt and eliminated the epitope for antibody B111. To learn if the seatbelts of these crosslinked analogs were attached to βCys26, their abilities to be recognized by antibody B111 and by B110 were compared, an antibody that recognizes a different β-subunit epitope. As shown in Table 2, each crosslinked heterodimer was recognized by B111, albeit not always as well as B110, an observation that suggested that the seatbelt was latched to βCys26 in the same manner as it is in hCG. While we cannot exclude the possibility that the βCys110 is latched to the cysteine that had been introduced into the α-subunit of some analogs, this seems highly unlikely for two reasons. First, all of the β-subunits used in these studies contain a cysteine at residue 26. Elimination of this cysteine at residue 26 is required to cause the seatbelt to become crosslinked to a cysteine introduced into the α-subunit regardless of their location (Xing, Y. Lin, W, Jiang, M., Myers, R. V., Cao, D., Bernard, M. P. & Moyle, W. R. Alternatively folded chorogonadotropin analogs: implications for hormone folding and biological activity. Journal of Biological Chemistry. 2001.) And second, the location of the α-subunit cysteine in the crosslinked analogs that were recognized least by B111 (αY37C, αP40C, αL41C, αR42C, and αT86C) is nearest the B111 binding site (FIG. 1). This suggests that the crosslink may have stabilized position of the β-subunit carboxyterminus of these analogs in a position that interfered with the access of B111 to the heterodimer.

Carboxyterminal End of The α-Subunit

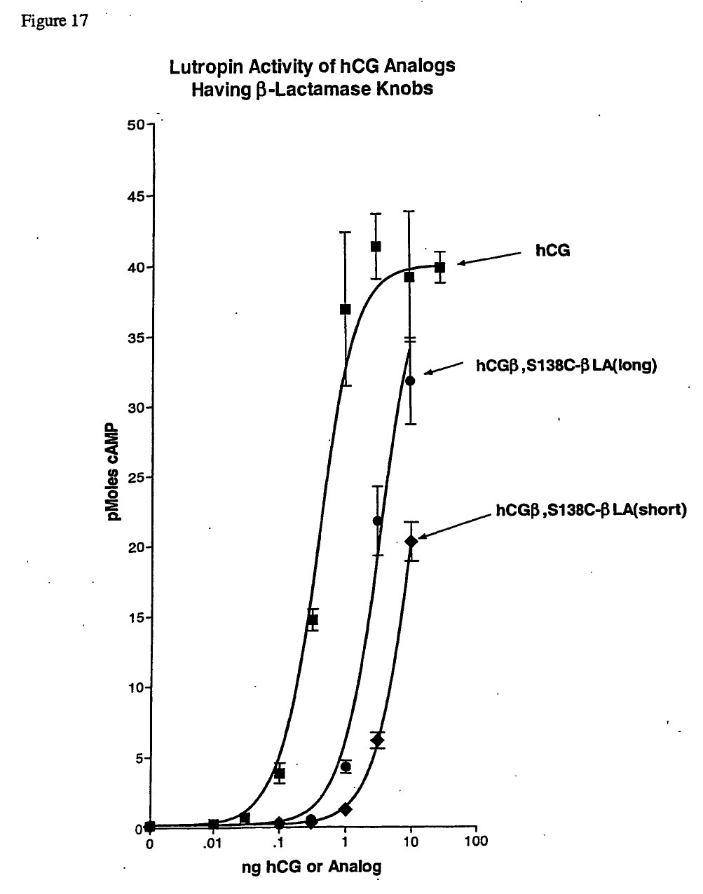

The carboxyterminal end of the α-subunit is thought to be essential for LHR interactions (Pierce, J. G. & Parsons, T. F. (1981) Annu. Rev. Biochem. 50, 465-495; Yen, S. S. C., Llerena, O., Little, B. & Pearson, O. H. (1968) J. Clin. Endocrinol. Metab. 28, 1763-1767.). The presence of cysteines in the carboxyterminal end of the α-subunit also led to a small reduction in activity of the heterodimer (Table 3, FIG. 3), a phenomenon consistent with the putative role of this portion of the hormone as a receptor contact (Pierce, J. G. & Parsons, T. F. (1981) Annu. Rev. Biochem. 50, 465-495; Chen, F., Wang, Y. & Puett, D. (1992) Mol. Endocrinol. 6, 914-919.) Nonetheless, these analogs were much more active than an analog lacking the five carboxyterminal α-subunit residues (Pierce, J. G. & Parsons, T. F. (1981) Annu. Rev. Biochem. 50, 465-495), an indication that no single residue in this region is essential for hormone activity and that each makes only a minor contribution to the total binding energy of hCG for the LHR. It was surprising to find that the heterodimer containing αK91C retained much of the efficacy of hCG (FIG. 3), since αLys91 has been reported to have an essential role in signal transduction (Yoo, J., Ji, I. & Ji, T. H. (1991) J. Biol. Chem. 266, 17741-17743) due to a putative contact that it makes with an aspartic acid residue in the transmembrane domain (Ji, I., Zeng, H. & Ji, T. H. (1993) J. Biol. Chem. 268, 22971-22974.) The finding that hCG-αK91C retained substantial efficacy is inconsistent with this proposal. As a result, hCG-αK91E and hCG-αK91M were prepared and their activities were tested. These analogs have been reported to have very low efficacy (Yoo, J., Ji, I. & Ji, T. H. (1991) J. Biol. Chem. 266, 17741-17743.) As shown in FIG. 17, both analogs had substantial efficacy, although the analog containing the glutamate interacted with LHR with approximately 10-fold lower affinity than hCG. This observation is consistent with observations made when αLys91 was converted to cysteine (Table 3, FIG. 3) and may indicate that this lysine residue is not as important for signal transduction as has been reported (Yoo, J., Ji, I. & Ji, T. H. (1991) J. Biol. Chem. 266, 17741-17743.).

Carboxyterminus of The β-Subunit and α-Subunit Loop 2

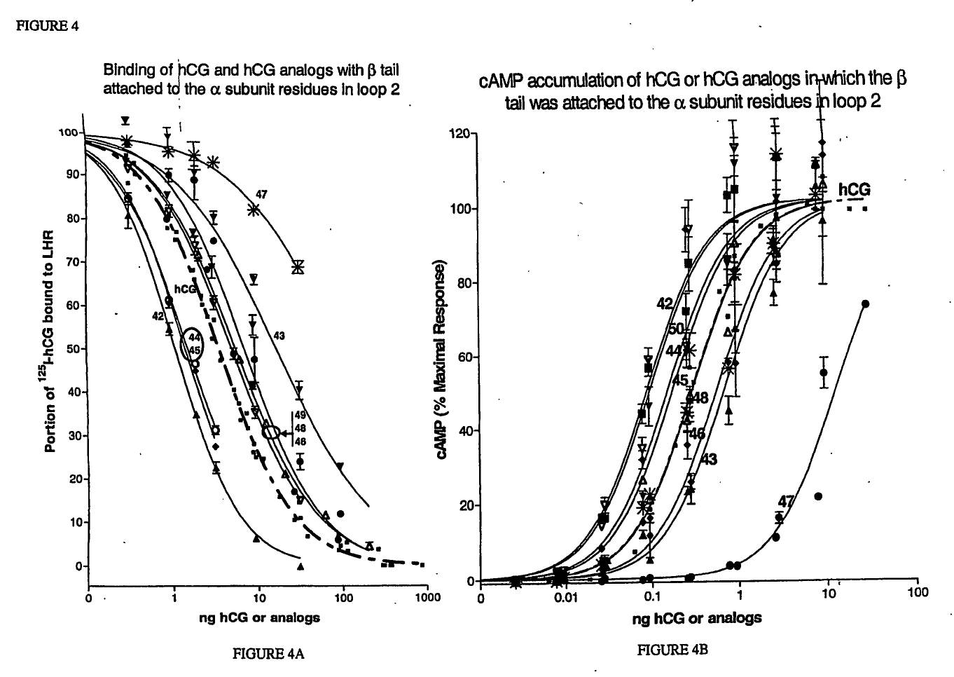

Many of the acid-stable analogs in which the carboxyterminus of the β-subunit was crosslinked to residues in α-subunit loop 2 had considerable activities in receptor-binding and signal-transduction assays (Table 3, FIG. 4). These include those in which it was attached to α-subunit loop 2 residues 35, 37, 40, 44, 42, 45, 50, and 52 (Table3). Attachment of the carboxyterminus of the β-subunit to α-subunit loop 2 residue 47 nearly abolished receptor binding activity and coupling it to residue 43 and 46 reduced the activity of the heterodimer by half. The sidechains of α-subunit loop 2 residues in nearly all the most active analogs project towards β-subunit loops 1 and 3. This suggests that the surface of α-subunit loop 2 that faces β-subunit loops 1 and 3 does not contact the LHR. The sidechains of α-subunit residues 43 and 46 project towards the seatbelt and that of residue 47 faces the small seatbelt loop formed by the disulfide between Cys93 and Cys100. Therefore, the loss in activity caused by linking the carboxyterminal portion of the β-subunit to these sites may disrupt interactions between these portions of α-subunit loop 2 with the receptor or alter the conformation of α-subunit loop 2 in a way that reduces the ability of the hormone to interact with the receptor.

The length of the β-subunit carboxyterminus suggests that residue 138 could be attached to residues in α-subunit loop 2 by passing over β-subunit loops 1 and 3 (FIG. 1). Thus, the carboxyterminal portion of the β-subunit might not occupy the space in the groove between α-subunit loop 2 and β-subunit loops 1 and 3 in any of these analogs. To study the activities of analogs in which this portion of the carboxyterminal portion of the β-subunit occupies this groove, analogs lacking D-subunit residues 116-135 and 121-135 were prepared. In these analogs, the β-carboxyterminus was too short to form a crosslink with α-subunit residues in loop 2 without passing through this intersubunit groove. These analogs had substantial activities in LHR binding and signaling assays (FIG. 4), providing additional support for the concept that this portion of α-subunit loop 2 does not contact the LHR.

β-Subunit and α-Subunit Loops 1 and 3

To learn if β-subunit residue 138 could become crosslinked to other portions of the α-subunit core, hCG-βS138C was expressed with several α-subunit analogs containing a cysteine in place of a residue in loops 1 or 3. With the exception of the heterodimer that contained a cysteine in place of αSer64, only small amounts of the resulting heterodimers were crosslinked as revealed by their low stability at acid pH (Table 2). The cysteines of many of the α-subunit analogs in these heterodimers were located much further from the carboxyterminal end of the seatbelt than those of analogs that participated in intersubunit crosslinks and, may have been beyond the range that was capable of being reached efficiently by the carboxyterminal region of the β-subunit (FIG. 1). The αS64C/hCG-βS138C heterodimer had considerable activity in receptor binding and signaling assays (Table 3), showing that this α-subunit residue does not make essential contacts with the LHR.

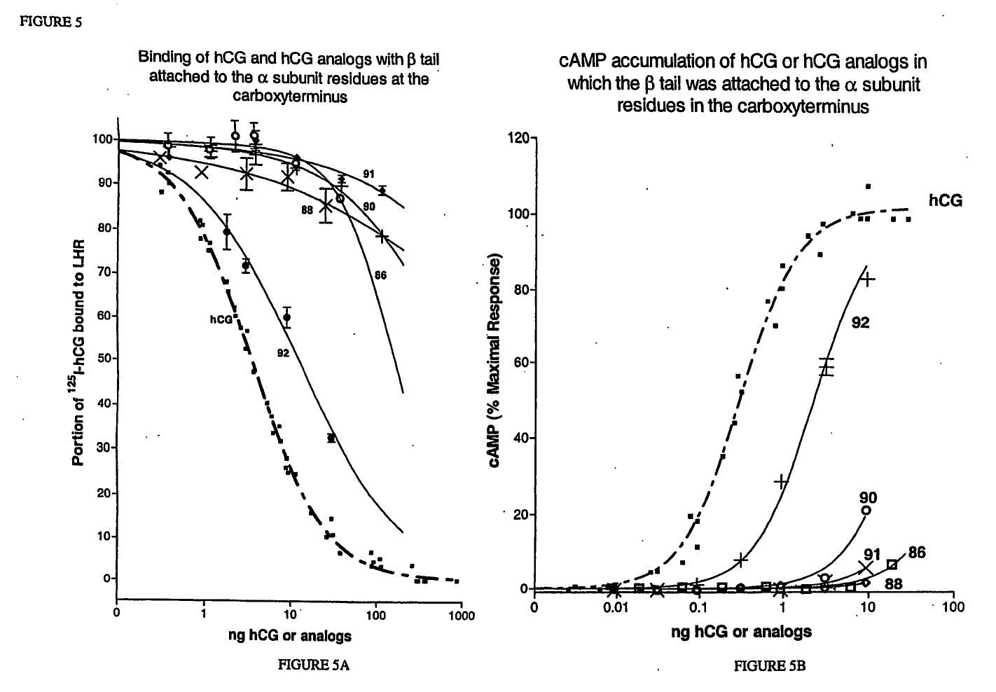

Crosslinking the β-subunit carboxyterminus to residues in the α-subunit carboxyterminus had a much larger influence on the interactions of the heterodimer with LHR than substitutions of cysteines in either subunit (FIG. 5). This suggested that the α-subunit carboxyterminus is near the LH receptor interface as had been proposed based on studies performed more than 25 years ago (Pierce, J. G. & Parsons, T. F. (1981) Annu. Rev. Biochem. 50, 465-495.) Nonetheless, it is possible that attaching the carboxyterminal end of the β-subunit to this region alters the structure of the heterodimer or causes the end of the β-subunit to pass near other portions of the hormone that contact the receptor.

Protein Knobs—hCG-βLactamase

These studies provide considerable support for the idea that most residues in α-subunit loop 2 do not participate in essential LH receptor contacts. The studies do not exclude the possibility that this portion of the hormone is near the receptor interface, however. To address this issue, hCG β-subunit analogs in which β-lactamase, a globular protein similar in size to hCG, was attached to specific sites on the α-subunit were prepared. This was accomplished by fusing β-lactamase to residues 140 and 145 of hCG-βS138C to yield hCG-βS138C-βLA140 and hCG-βS138CβLA145, respectively. The latter analog had a “spacer” of seven residues between the cysteine that was to be crosslinked to the α-subunit and the aminoterminus of β-lactamase. Co-expression of these β-subunit analogs with α-subunit analogs αT46C, αL48C, αS64C, and αS92C led to the formation of acid stable heterodimers (FIG. 6). This showed that the presence of the β-lactamase did not prevent the β-subunit residue 138 from becoming attached to the α-subunit.

These hCG-β-lactamase analogs had much lower biological activities than the corresponding analogs that lacked the β-lactamase (FIG. 6).

In conjunction with results of earlier studies designed to identify portions of the α-subunit that are not involved in key receptor contacts (Xing, Y., Lin, W., Jiang, M., Myers, R. V., Cao, D., Bemard, M. P. & Moyle, W. R. Alternatively folded choriogonadotropin analogs: implications for hormone folding and biological activity. Journal of Biological Chemistry. 2001.), these observations expand the present knowledge of the surfaces of the hormone that do not appear to contact the LHR and suggest that the groove between α-subunit loop 2 and β-subunit loops 1 and 3 does not participate in essential LH receptor contacts, a key requirement of an earlier model (Moyle, W. R., Campbell, R. K, Rao, S. N. V., Ayad, N. G., Bemard, M. P., Han, Y. & Wang, Y. (1995) J. Biol. Chem. 270, 20020-20031.) Indeed, based on the abilities of monoclonal antibodies to recognize hCG and hCG analogs bound to LHR on the surface of cells (Wang, Y. H., Bernard, M. P. & Moyle, W. R. (2000) Mol. Cell. Endocrinol. 170, 67-77) and the abilities of hCG/hFSH and hCG/hTSH chimeras to bind LHR (Campbell, R. K., Dean Emig, D. M. & Moyle, W. R. (1991) Proc. Natl. Acad. Sci. (U.S.A.) 88, 760-764; Campbell, R. K., Bergert, E. R., Wang, Y., Morris, J. C. & Moyle, W. R. (1997) Nature Biotech. 15, 439-443,; Grossmann, M., Szkudlinski, M. W., Wong, R., Dias, J. A., Ji, T. H. & Weintraub, B. D. (1997) J. Biol. Chem. 272, 15532-15540; Moyle, W. R., Campbell, R. K, Myers, R. V., Bemard, M. P., Han, Y. & Wang, X (1994) Nature 368, 251-255), it appears that few hCG-specific residues participate in essential LHR contacts. Residues that have the greatest influence on hCG activity are located in the seatbelt, but even most of these can be altered one-by-one without disrupting hormone-receptor interactions more than a few fold (Han, Y., Bemard, M. P. & Moyle, W. R. (1996) Mol. Cell. Endocrinol. 124, 151-161.) Based on these observations, the finding that two distant sites of the receptor appear to influence hCG-LHR interactions Bemard, M. P., Myers, R. V. & Moyle, W. R. (1998) Biochem. J. 335, 611-617, and the finding that mammalian lutropin interactions with human LHR are particularly sensitive to small changes in the conformation of the hormone, it is proposed that interactions of the glycoprotein hormones and their receptors are not dominated by relatively few contacts, such as those found between growth hormone; and its receptor (Clackson, T. & Wells, J. A. (1995) Science 267, 383-386; Wells, J. A. (1996) Proc. Natl. Acad. Sci. (U.S.A.) 83, 1-6.)

### EXAMPLE 2

### Protein Knobs to Study Docking of hFSH to FSH Receptors

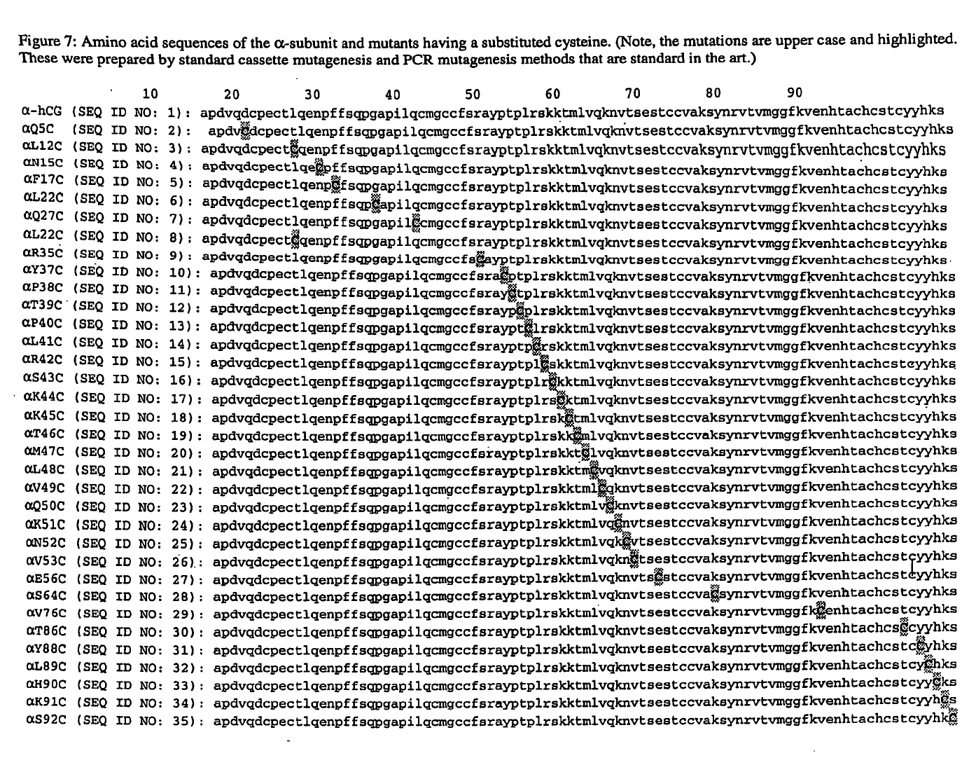

A cDNA coding sequence for the hFSH β-subunit was obtained from Christie Kelton, Ares Advanced Technology, a division of Serono, 280 Pond Street, Randolph Mass. that corresponds to the translated portion of the mRNA. The amino acid sequence of this β-subunit lacking the leader peptide is illustrated in (FIG. 8, SEQ ID NO: 40). The hFSH cDNA was modified by PCR and cassette mutagenesis to create a construct termed FC1-108β (FIG. 8, SEQ ID NO: 41) that encodes hFSH residues 1-108 and hCG residues 115-145 in tandem. The amino acid sequence of FC1-108β lacking the leader peptide found in hFSH β-subunit is also illustrated in FIG. 8. An analog of FC1-108β in which residue Ser132 was converted to Cys was prepared by replacing the Xhol-Apal fragment of the construct that encodes hCG-S138Cβ with that of FC1-108β to create FC1-108,S132Cβ (FIG. 8, SEQ ID NO: 42). FC1-108,S132C was expressed with several of the α-subunit analogs shown in FIG. 7 in COS-7 cells using methods described in Example 1. The heterodimers secreted by the cells into the culture media were measured using antibodies A113 and ¹²⁵I-B603. The former is an antibody to the α-subunit and the latter is a monoclonal antibody that binds to hFSH β-subunit. Purified hFSH was used as a standard. Following acid treatment to dissociate any non-crosslinked heterodimer, the material was re-assayed with A113 and ¹²⁵l-B603 as above to determine the amount of crosslinked material in the sample. The resulting crosslinked analogs were tested for their abilities to elicit cyclic AMP accumulation in CHO cells that express FSH receptors.

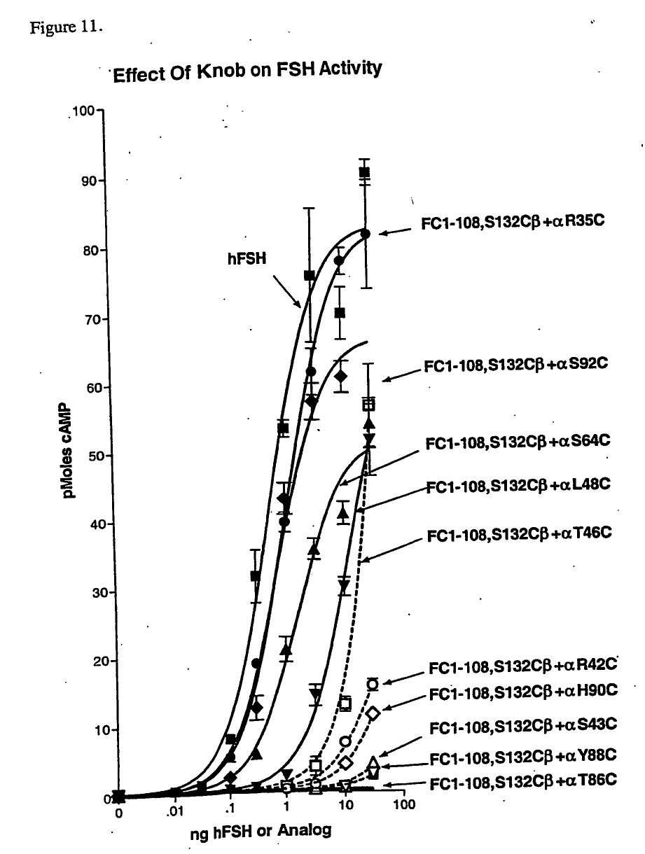

As shown in FIG. 11, the influence of the knob at α-subunit residue 35 resulted in only a modest decrease in the activity of this FSH analog. Addition of the knob to α-subunit residues 92, 64, 48, 46, 42 90, 43, 88, and 86 resulted in a progressive loss in analog activity. This showed that the α-subunit residue 35 is not near the FSH receptor interface and that several other residues appear to be near the receptor interface. Unlike the interactions of hCG with the LH receptor, the presence of a knob at residues 42 and 43 were much more inhibitory to the binding of the FSH analog to its receptor. This revealed that the surface of part of α-subunit loop 2 was much closer to the FSH receptor than to the LH receptor when FSH and hCG interact with their respective receptors. The finding that attaching a knob to α-subunit residue 86 of both receptors showed that this portion of both ligands is near the receptor interface.

### EXAMPLE 3

### Protein Knobs to Study Docking of Bifunctional hCG/hFSH Cimera to LH and FSH Receptors

A cDNA sequence encoding a chimera in which hCG β-subunit codons for amino acids 101-114 were replaced with their hFSH β-subunit counterparts was prepared by standard methods. The amino acid sequence of this chimera termed CFC101-114β is shown in FIG. 8 (SEQ ID NO: 38) minus the leader residues. An analog of CFC101-114β in which residue Ser138 was converted to Cys was prepared by replacing the Xhol-Apal fragment of the construct that encodes hCG-S138Cβ with that of CFC101-114β to create CFC101-114β, S138C (FIG. 8, SEQ ID NO: 39). CFC101-114β, S138C. was expressed with several of the α-subunit analogs shown in FIG. 7 in COS-7 cells using methods described in Example 1. The heterodimers secreted by the cells into the culture media were measured using antibodies A113 and ¹²⁵I-B110. The former is an antibody to the α-subunit and the latter is a monoclonal antibody that binds to hCG β-subunit. Purified hCG was used as a standard. Following acid treatment to dissociate any non-crosslinked heterodimer, the material was re-assayed with A113 and ¹²⁵I-B110 as above to determine the concentration of crosslinked material in the sample. The resulting crosslinked analogs were tested for their abilities to inhibit binding of ¹²⁵l-hCG to CHO cells expressing LH receptors and to inhibit the binding of ¹²⁵l-hFSH to CHO cells expressing FSH receptors (FIG. 12). They were also tested for their abilities to elicit cyclic AMP accumulation in CHO cells that express LH receptors and in CHO cells that express FSH receptors.

As shown in FIG. 12, the presence of a knob at α-subunit residue 35 did not interfere with the ability of the chimera to block binding of radioiodinated hCG or radioiodinated hFSH to the LH or FSH receptors, respectively relative to a bifunctional chimera lacking most of the hCG β-subunit carboxyterminus. The presence of a knob at α-subunit residue 37 led to a dramatically different result, however. Thus, this knob had very little influence on the ability of the chimera to bind to LH receptors but nearly eliminated its ability to bind to the FSH receptors. The presence of a knob at α-subunit residues 43 and 46 reduced the abilities of the chimera to bind both receptors. As was found for hCG binding to LH receptors and hFSH binding to FSH receptors, the presence of the knob at these sites was much more inhibitory to FSH receptor interactions than LH receptor interactions. This showed that these residues were much closer to the FSH receptor interface than the LH receptor interface. The presence of the knob at α-subunit residues 48 and 52 had much less influence on interactions of the chimera with either receptor, indicating that these residues are further from the receptor interface. As had been observed in examples 1 and 2, the presence of a knob at α-subunit residue 86 was very inhibitory to binding of the chimeras to both receptors. This showed that this portion of the hormone is near the interface of the chimera with both receptors. A similar result was observed with the knob at α-subunit residue 91.

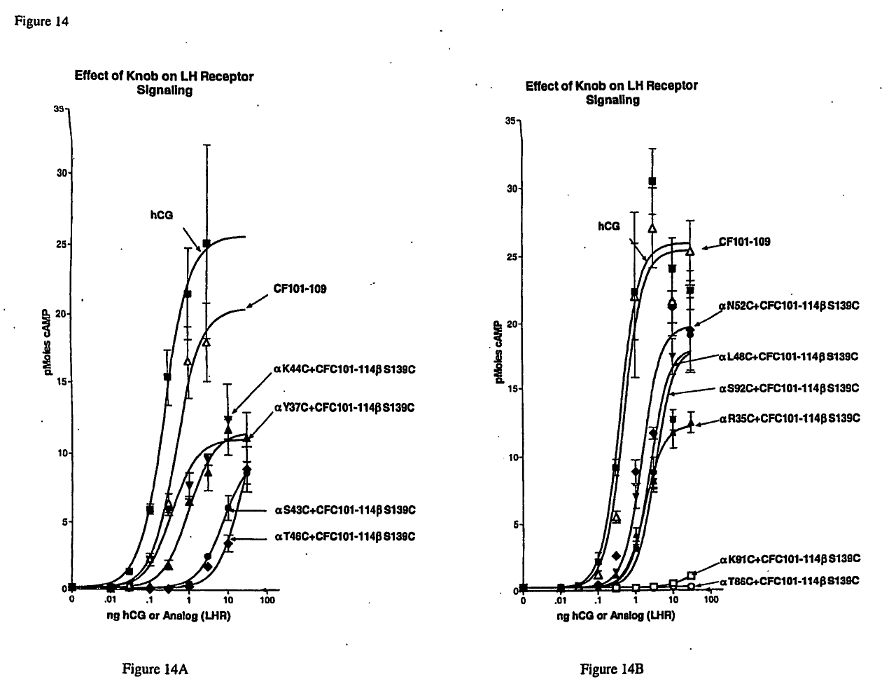

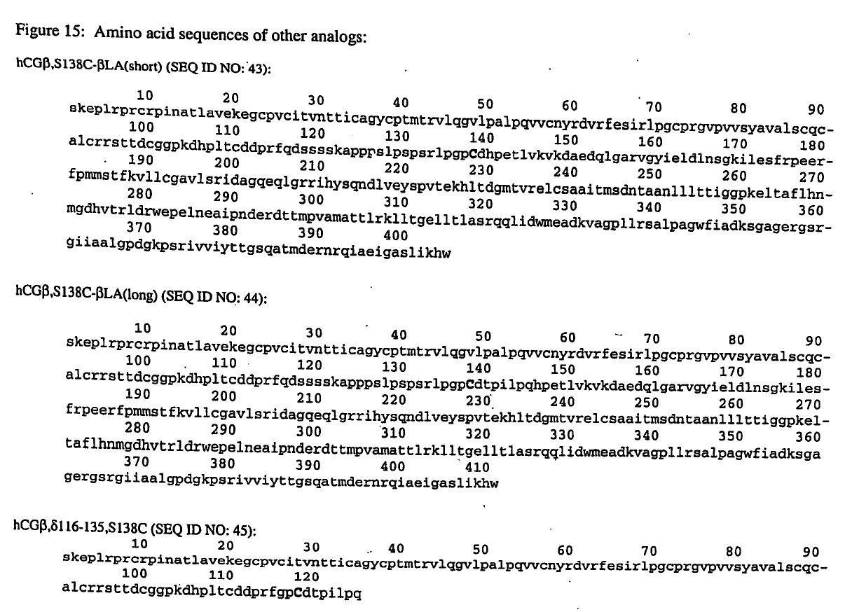

As can be seen in FIGS. 13 and 14, the presence of a knob reduced the activity of most chimeras in LH receptor and FSH receptor signal transduction assays. The amount of reduction differed, however, depending on the receptor with which the analog was being tested. For example, the presence of a knob at α-subunit residue 37 was much more inhibitory on FSH receptor elicited cyclic AMP accumulation than on LH receptor mediated cyclic AMP accumulation. Overall, the relative influence of the knobs on the abilities of the chimeras to elicit a cellular response was similar to their abilities to affect receptor interactions. These studies led to the conclusion that the interactions of lutropins such as hCG with LH receptors were readily distinguished from those of follitropins such as hFSH with FSH receptors. Thus, even though lutropins and follitropins have very similar structures and even though their receptors are quite similar, the manner in which these ligands were found to interact with the receptors were not identical. This example illustrates the power of using the probes of the present invention in identifying protein-protein interactions.

### EXAMPLE 4

### Knobs having β-Lactamase

Analogs containing hCGβ, S138C β-subunits have relatively small knobs consisting of residues surrounding Ser138. These are sufficiently large to detect distances between the ligand and its receptor that are relatively small. However, these knobs are too small to be useful for detecting the proximity of residues that are further from the receptor. This limitation can be circumvented by increasing the size of the probe, as was done by adding β-lactamase. β-lactamase was chosen for use as a probe because its crystal structure is known and because its aminoterminal and carboxyterminal ends are on the surface of the protein. Thus, it is useful for generating fusion proteins at either end of the hormone. Another advantage of β-lactamase is that it is an enzyme with a high turnover number that cleaves fluorescent substrates (Zlokamik, G., Negulescu, P. A., Knapp, T. E., Mere, L., Burres, N., Feng, L., Whitney, M., Roemer, K. & Tsien, R. Y. (1998) Science 279, 84-88), a phenomenon that would be useful for detection of the fusion protein. In this regard β-lactamase has been used as an efficient reporter (Moore, J. T., Davis, S. T. & Dev, I. K (1997) Anal. Biochem. 247, 203-209), albeit in a very different context than is envisioned here, β-lactamase is also inhibited by a protein that binds to it (Strynadka, N. C., Jensen, S. E., Johns, K., Blanchard, H., Page, M., Matagne, A., Frere, J. M. & James, M. N. (1994) Nature 368, 657-660.) Thus, it would be possible to increase the size of the β-lactamase knob further simply by adding the inhibitor.

Two derivatives of hCGβ,S138C were prepared that contained a β-lactamase fusion protein. One, known as HCGβ,S138-βLA(long) (SEQ ID NO: 44) or more simply LONG, contains β-lactamase fused to the carboxyterminal end of hCGβ,S138C as shown in FIG. 15. This protein was prepared by PCR mutagenesis using pUC18 as a template. The other probe known as hCGβ,S138C-βLA(short) (SEQ ID NO: 43) or more simply SHORT, contains β-lactamase fused to a truncated version of hCGβ,S138C as shown in FIG. 15. The SHORT protein has only one amino acid between the coupling cysteine and the start of the β-lactamase. Clearly, other versions of these could be made in which a different number of residues are placed between the coupling cysteine and the β-lactamase. It should also be recognized that one skilled in the art of molecular modeling and molecular biology could design and engineer an analog of β-lactamase in which a surface residue was replaced by a cysteine. This cysteine could serve as the coupling cysteine, a phenomenon that would hold the β-lactamase knob in a more rigid connection to the target cysteine than would be possible using a tail.

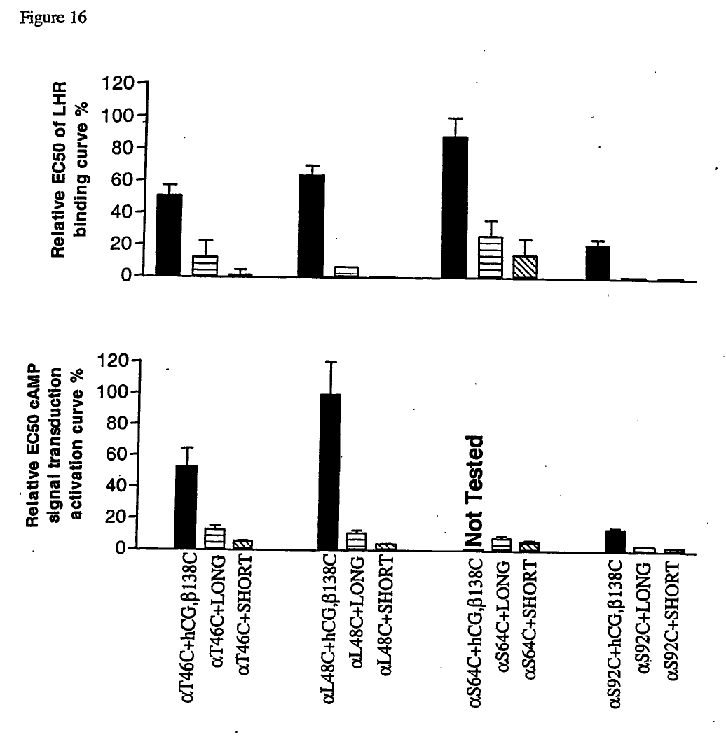

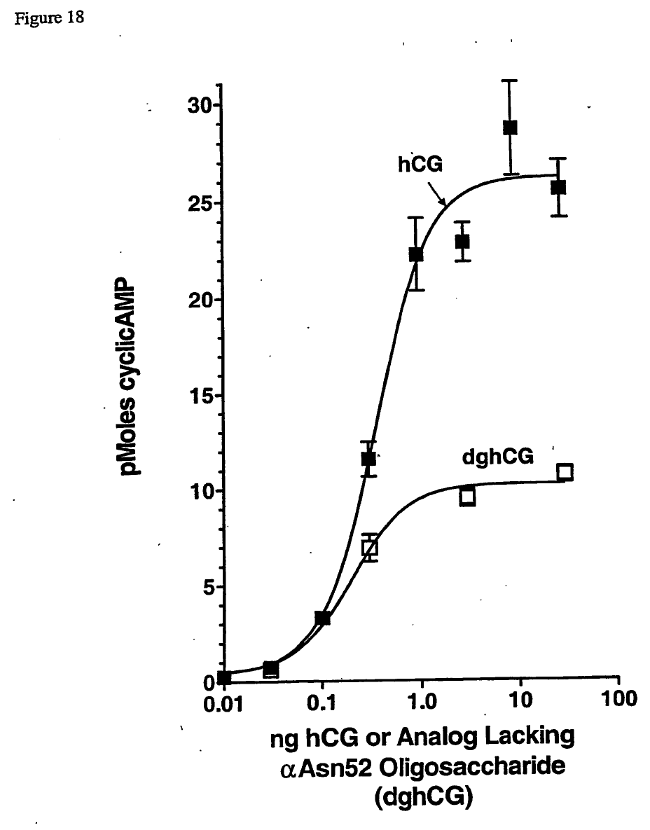

Five different acid stable heterodimers were produced containing the LONG and SHORT β-subunits. These were made to determine the relative proximity of α-subunit residues 46, 48, 52, 64, and 92 to the LH receptor. As shown in FIGS. 16 and 17, none of these analogs were as active as hCG. Heterodimers in which the LONG and SHORT β-subunit knot probes were coupled α-subunit residue 52 [i.e., αN52C+hCGβ,Si38C-βLA(long) and αN52C+hCGβ,S138C-βLA(short), respectively] retained much of the activity of hCG in signal transduction assays. Changing α-subunit residue Asn52 to cysteine disrupts a glycosylation signal and results in the loss of an oligosaccharide on α-subunit loop 2. Removal of this oligosaccharide from hCG causes it to lose approximately 60% of its efficacy (FIG. 18). Thus, addition of the β-lactamase knob to this site can offset the loss in efficacy caused by removal of the oligosaccharide from loop 2. The relatively high activity of both these LONG and SHORT analogs show that. α-subunit residue is not near the receptor interface. Since both analogs have high efficacy, these findings have implications for the role of the oligosaccharide in hCG-induced signal transduction. The activities of the LONG and SHORT analogs indicate that the role of the oligosaccharide on α-subunit loop 2 is to distort the positions of the subunits within hCG; a phenomenon that does not depend on specific contacts between the oligosaccharide and either subunit.

The activities of the remaining LONG and SHORT analogs show that α-subunit residues 48 and 64 are much closer to the receptor than would have been determined from the activities of hCGβS138C+αL48C and hCGβS138C+αS64C. This discovery has important consequences for models of the hCG-LH receptor complex. It also confirms the earlier findings that α-subunit residues 46 and 92 are close to the receptor interface.

### EXAMPLE 5

### Knobs Having a Coupling Cysteine at the Aminoterminal End of the α-Subunit

It is not necessary for the coupling cysteine to be at the carboxyterminal end of the protein and analogs have been designed in which the coupling cysteine is at the aminoterminal end of the protein. The amino acid sequence of one such analog (β101-145, α) is illustrated below. This analog was prepared by deleting hCG β-subunit residues 3-100 and fusing the α-subunit to the end of the remaining β-subunit using standard PCR and cassette mutagenesis methods. It has a free cysteine at residue 12 that serves as the coupling cysteine.

```
β101-145, α
(SEQ ID NO: 54)
                10 20 30 40 50 60
skggpkdhpltcddprfqdsssskapppslpspsrlpgpsdtpilpqapdvqdcpectlq-
                70 80 90 100 110 120
enpffsqpgapilqcmgccfsrayptplrskktmlvqknvtsestccvaksynrvtvmgg-
              130 140
fkvenhtachcstcyyhks
```

### EXAMPLE 6

### Method for Producing Site-Specific Protein Knobs

A protein to which a knob is to be attached is selected. Then, the specific location to be labeled on the protein is determined. Using mutagenesis techniques known in the art, a construct capable of expressing the protein may be prepared such that a cysteine residue is substituted for the native residue at the specific location of labeling in the encoded protein. In addition, the protein encoded in the construct will further comprise a tail portion which contains a cysteine residue attached to one end of the protein, and a knob attached to the end of the tail portion. The construct is then inserted into a cell for expression resulting in the production of a protein with a knob attached at the specified site.

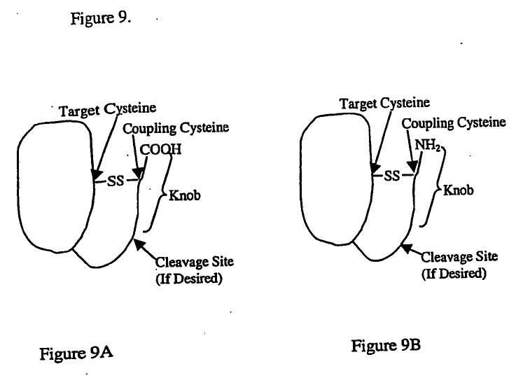

Addition of a knob to a protein involves introducing a cysteine onto the surface where the knob is to be located and introducing a cysteine at a site in the aminoterminal or carboxyterminal end of the protein such that the cysteine in the end of the protein can form a disulfide with that that has been added to the surface. Formation of a disulfide between these two cysteines will cause the end of the protein containing the cysteine to be stabilized at the surface creating a knob at this site (see FIG. 9).

In order for the knob to be created, the end of the protein that contains the cysteine should be of sufficient length to permit its cysteine to form a disulfide with the cysteine at the site that will contain the knob. This may require addition of a tail portion such as the carboxyterminal end of the hCG β-subunit as was the case for addition of a protein knob to hFSH. The composition of the tail portion can be varied extensively and need not be limited to that of the hCG β-subunit. The major requirements of the tail portion are that it should be sufficiently long to reach the cysteine on the surface of the protein that defines the location of the knob and that it lack residues that prevent it from getting to that site. These would include residues characteristic of transmembrane domains and residues that create sites that bind to other proteins or to a part of the protein distant from the cysteine on the surface of the protein at the knob.

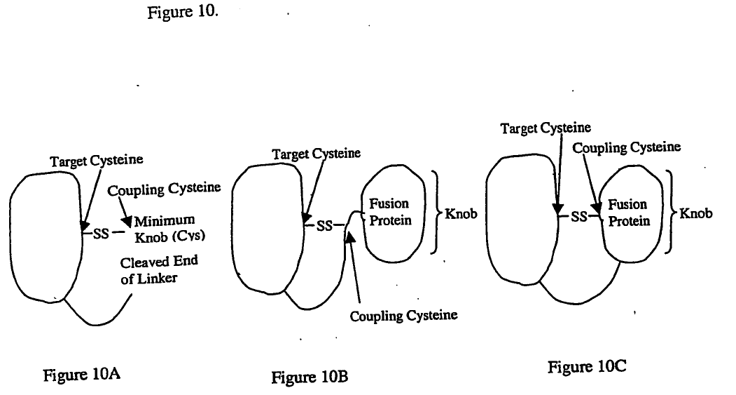

The residues on either side of the cysteine that is in the aminoterminal or carboxyterminal end of the protein form the knob. (See FIG. 9). In general, the greater the number of residues at this site, the larger the knob. The smallest knobs would consist only of a cysteine. (See FIG. 10A). These would be created by incorporating a cysteine at the end of the aminoterminal or carboxyterminal end of the linker and adding a cleavage site immediately adjacent to the cysteine. (See FIG. 10A). The cleavage site should contain an amino acid sequence for a protease that is not found within other parts of the protein. The size of the knob can be increased in at least three fashions. First, a protein can be fused to the end of the protein used to construct the knob. (See FIG. 10B). As described above, β-lactamase was fused to the end of the protein. However, β-lactamase is not the only protein that is suitable for this purpose. The choice of β-lactamase as a probe was facilitated by its crystal structure, which showed that its aminoterminal end was located on its surface, a position favorable for construction of a fusion protein. Second, cleavage of the linker by a protease can be used to reduce the size of the linker and its contribution to the knob. And third, the distance between the coupling cysteine and the fusion protein probe can be varied by increasing or reducing the number of amino acids. As seen for the hCG example, a shorter distance caused a greater reduction in hormone activity due to the fact that it restricted movements of the knob. This kept the knob closer to the interface between the hormone and the LH receptor.

As should be evident from FIG. 10, it is not essential for the coupling cysteine to be present in the tail portion. Introduction of a cysteine in the surface of the fusion protein that is part of the knob where it can form a disulfide with the target cysteine on the protein should cause the fusion protein to become attached directly to the surface of the protein to be probed. (See FIG. 10C). This method can also be used to control the orientation of the fusion protein that is part of the knob portion. When proteases are used as fusion proteins, introduction of cysteines into a surface distant from the active site will keep the active site of the fusion protein from the surface to which the knob is being attached.

### EXAMPLE 7

### Use of Shortened Tail Portion as the Probe

Addition of knobs to proteins permits the estimation of distances between two protein surfaces. As shown, substantial portions of α-subunit loop 2 of hCG do not contact the receptor. The location of the coupling cysteine at amino acid 138 of the hCG β-subunit places relatively few constraints on the positions that can be occupied by β-subunit residues 111-137 that link the coupling cysteine on the knob to the core of the hCG β-subunit. The tail portion itself can also be used to probe the surface of proteins and there are several instances in which it would be desirable to constrain its position to particular portions of the molecule. These include those in which it would be desirable to cover a portion of an active site of a protein, thereby rendering it inactive until the tail portion is clipped by a protease.

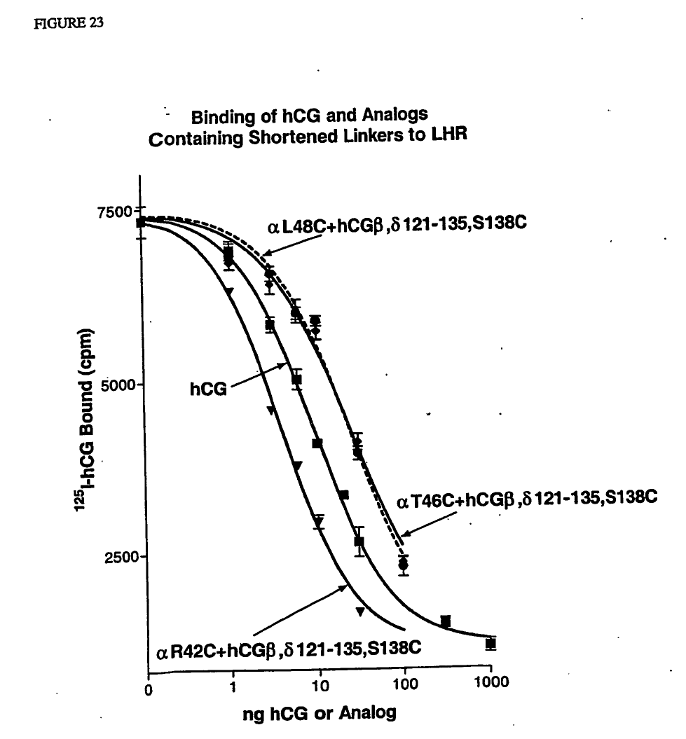

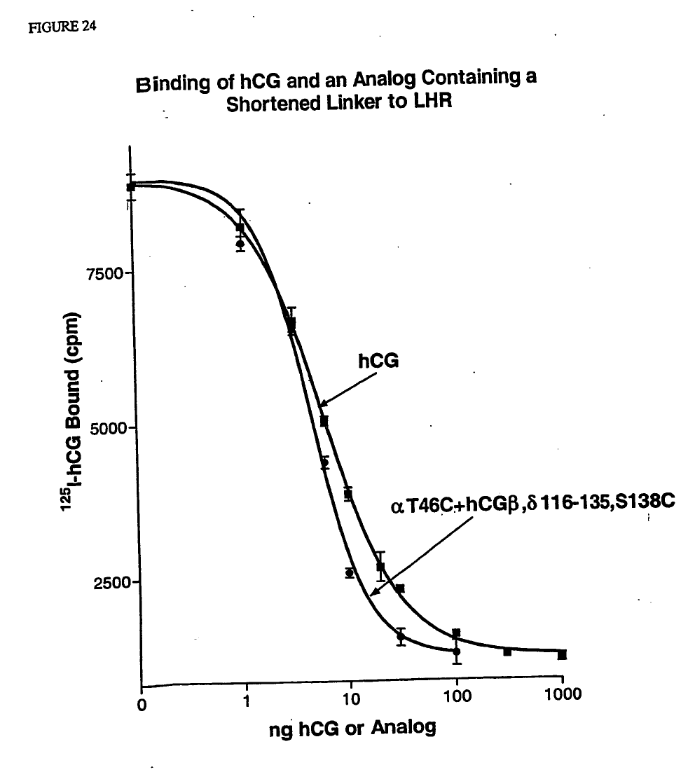

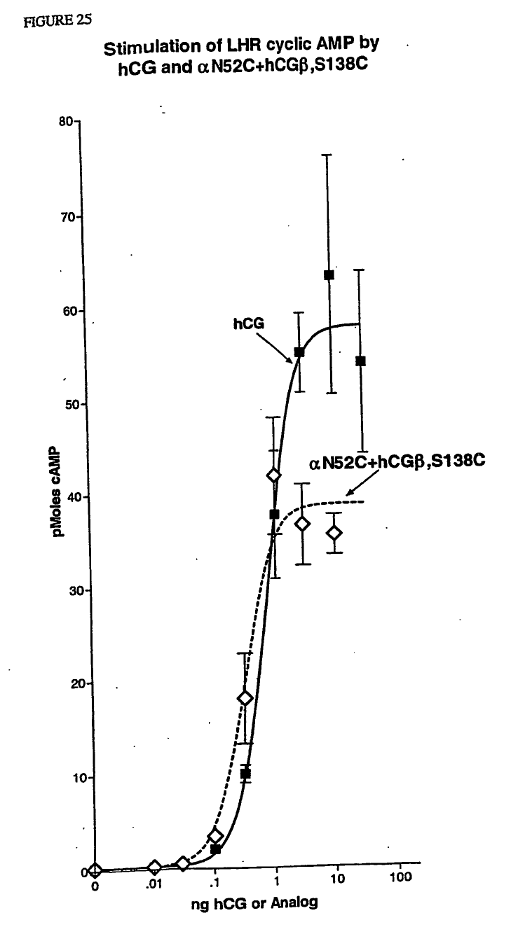

In studies of hCG-LH receptor interactions, it was desirable to constrain the location of the tail portion to the groove between α-subunit loop 2 and β-subunit loops 1 and 3, in order to test the assertion that this groove formed a key receptor contact. The use of the tail portion described earlier would permit it to pass through this portion of the hormone but it would not force it to be located there. Due to its length, the tail portion might have passed over the convex surface of β-subunit loops 1 and 3 to enable the knob coupling cysteine to reach various cysteines that had been substituted for residues in α-subunit loop 2. Therefore, we shortened the tail portion to force it to pass through the groove between α-subunit loop 2 and β-subunit loops 1 and 3 when targeting the cysteine that had been substituted for residues 42, 46 or 48 of the α-subunit. The tail portion analogs hCGβ,δ116-135,S138C (SEQ ID NO: 45) and hCGβ,δ121-135,S138C (SEQ ID NO: 46(FIG. 15) were too short to enable the formation of a disulfide between the knob and the target protein unless the tail portion analogs passed through the groove between α-subunit loop 2 and β-subunit loops 1 and 3. As can be seen from the results shown in FIG. 23 and 24, heterodimers containing αT46C and hCGβ,δ116-135,S138C or HCGβ,δ121-135,S138C had substantial receptor binding activity. The heterodimer containing αL48C and HCGβ,δ121-135,S138C also had considerable receptor binding activity. Much smaller amounts of acid stable heterodimer containing αL48C and hCGβ,δ116-135,S138C were formed, indicating that the shortest tail portion tested may not have been of sufficient length for the knob coupling cysteine to reach the target cysteine on the protein. Since it was not necessary for the tail portion of these analogs to pass through the groove between α-subunit loop 2 and β-subunit loops 1 and 3 for the knob coupling cysteine to reach the target cysteine at α-subunit residue 42, this analog was used as a positive control.

Taken together, these observations showed that the groove did not participate in essential receptor contacts. It also demonstrated how the position of the tail portion can be manipulated to enable it to pass near specific portions of the protein. This would permit the tail portion to be used to obscure a specific site, a property that when combined with cleavage sites that have been introduced into the tail portion would be useful for preparing latent proteases, toxins, or other useful analogs. Tail portions that obscures a protease or toxin site would permit preparation and use of reagents capable of entering cells where endogenous or other proteases would cleave the tail portion to activate the toxin. These agents would be useful as therapeutics to treat malignancies or other diseases.

### EXAMPLE 8

### Use of Probes Attached to the β-Subunit

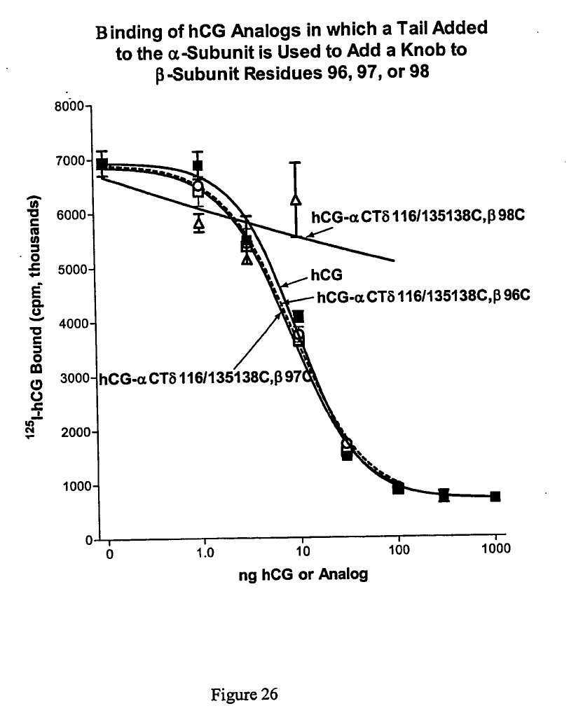

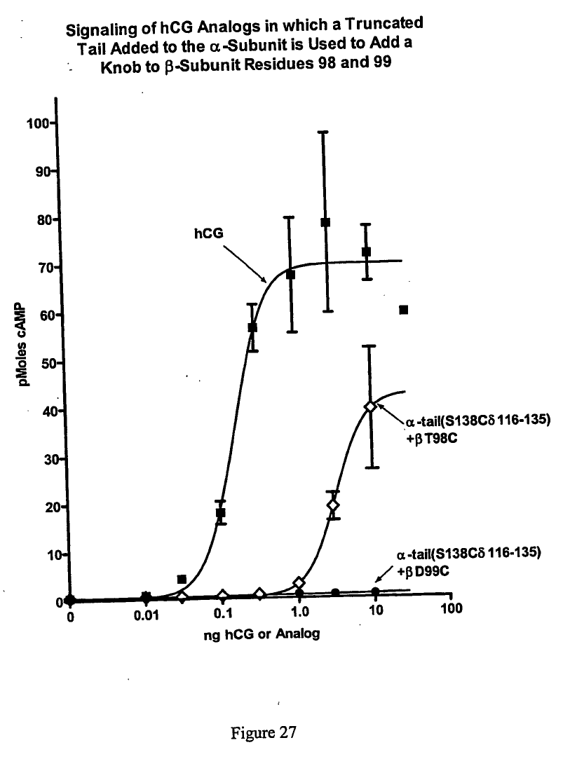

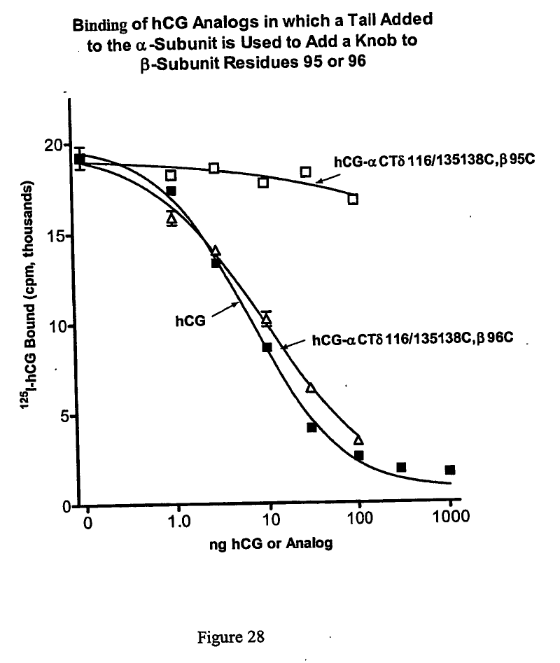

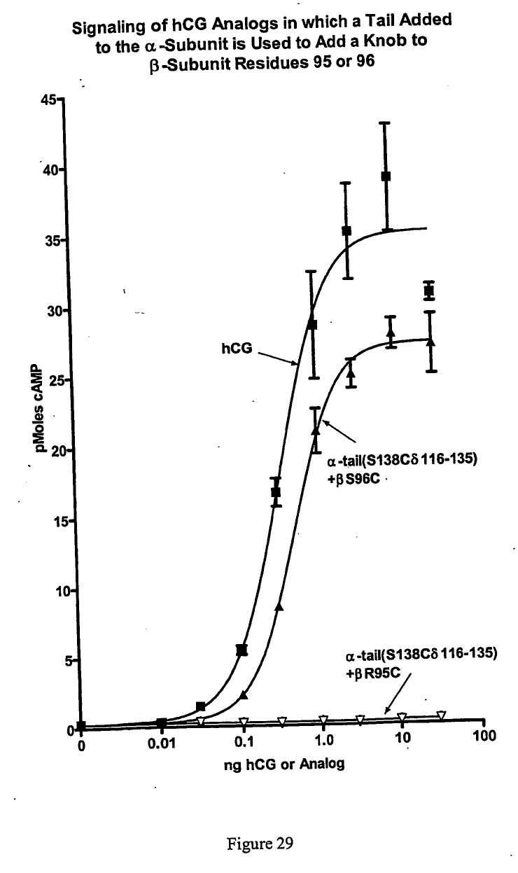

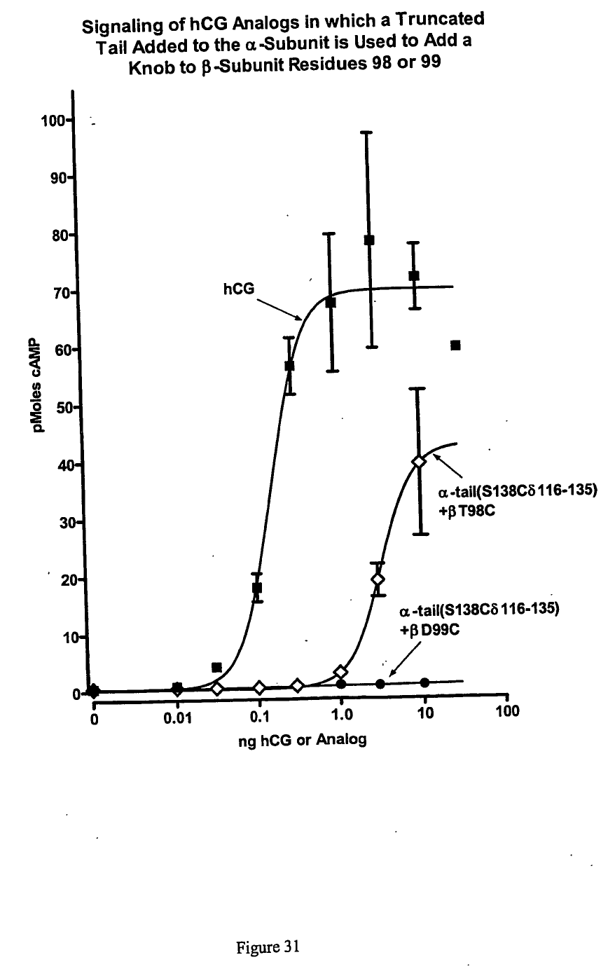

The small loop in the seatbelt has been suggested to have a role in the biological activity of hCG and it was desired to attach knobs to this region of the β-subunit to investigate this possibility. Analogs of the alpha subunit were produced that contained the carboxyterminal residues in the hCG β-subunit. While it has been shown that the attachment of a portion of the carboxyterminus of the hCG β-subunit to the carboxyterminal end of the α-subunit reduced the activity of the heterodimer by 50-fold or more, the use of the entire β-subunit sequence had no such consequence. Analogs containing the entire carboxyterminal residues of the hCG β-subunit attached to the α-subunit, including amino acids Asp-Asp-Pro-Arg-Phe-Gln-Asp-Ser-Ser-Ser-Ser-Lys-Ala-Pro-Pro-Pro-Ser-Leu-Pro-Ser-Pro-Ser-Arg-Leu-Pro-Gly-Pro-Ser-Asp-Thr-Pro-lle-Leu-Pro-Gln, had 50% or more the activity of hCG in receptor binding and signaling assays. This was found to be due to the presence of charged residues near the junction of the residues derived from the α-subunit and the β-subunit. To attach a knob to β-subunit residues in the small seatbelt loop, a truncated version of the hCG β-subunit carboxyterminus was added to the end of the α-subunit to create hCG-αCTδ116/135,138C (SEQ ID NO: 57). This was co-expressed in COS-7 cells with a hCG β-subunit analog having a cysteine in place of Arg94 (SEQ ID NO: 58), Arg95 (SEQ ID NO: 59), Ser96 (SEQ ID NO: 60), Thr97 (SEQ ID NO: 61), Thr98 (SEQ ID NO: 62) and Asp99 (SEQ ID NO: 63). All of these proteins formed acid-stable heterodimers and as can be seen from the data in FIGS. 26, 27, 28, 29, 30, 31, and 32, these studies revealed how this part of the β-subunit interacts with LH receptors (LHR). The presence of this knob at residues 95 and 99 eliminated LHR interaction and biological activity. This knob had much less influence when present at residues 96 or 97 as these analogs had substantial activity in binding and signaling assays. The knob reduced receptor interaction more when it was attached to residue 98 but not nearly to the level seen when it was attached to residues 95 or 99. This suggests the latter residues may be located nearer the receptor interface.

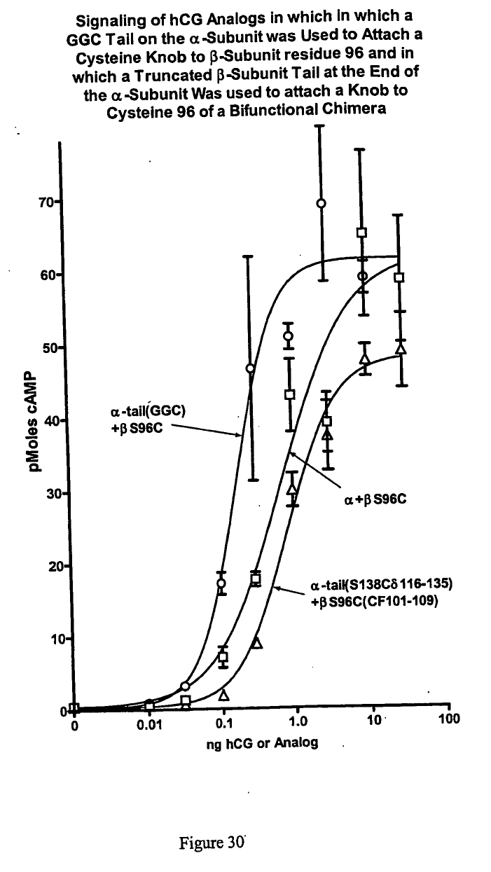

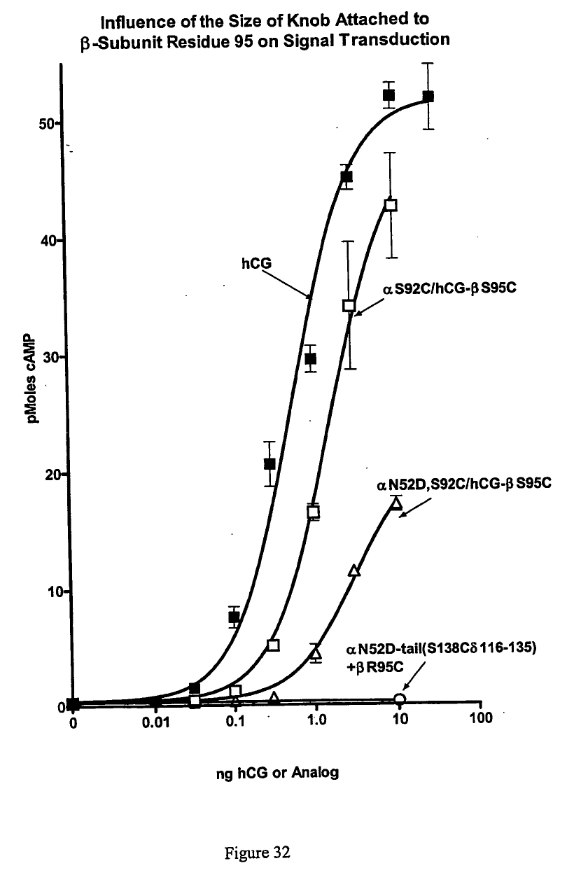

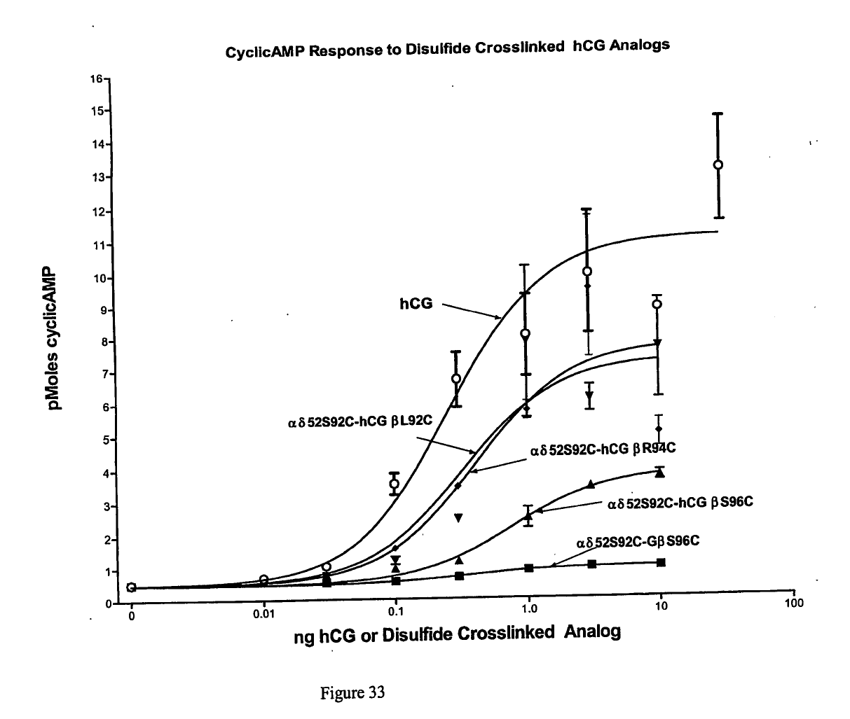

The sidechain of hCG β-subunit residue 95 faces in an opposite direction from those of residues 94 and 96, a phenomenon that may account for the fact that attaching a knob to this site was much more inhibitory to hCG-LHR interactions than attaching a knob at residues 94 and 96. This suggested that the surface of the seatbelt near the sidechain of Arg95 might be near the receptor interface. To learn if the sidechain contacted the surface of the protein, analogs were prepared that contained smaller knobs. These were constructed by replacing the serine in the at α-subunit residue 92 with cysteine (SEQ ID NO: 35) or by adding a tail composed of Gly-Gly-Cys to the carboxyterminus of the α-subunit (SEQ ID NO: 64). As can be seen in FIGS. 30 and 32, these smaller knobs had much less ability to interfere with the activity of the heterodimer in LHR assays. Thus, both acid-stable crosslinked heterodimers retained considerable activities in these assays. This showed that the sidechain of β-subunit residue Arg95 is likely to be near the receptor interface but not needed for essential receptor contacts. This also showed that the location of the carboxyterminal end of the α-subunit is near the small seatbelt loop after the hormone interacts with the LHR.

Since the α-subunit carboxyterminus is needed for full glycoprotein hormone activity, as is the oligosaccharide on α-subunit loop 2 at Asn52, it was of interest to see determine how changing the position of the α-subunit carboxyterminus in the heterodimer influenced its biological activity. An analog of hCG α-subunit that cannot be glycosylated at α-subunit loop 2 due to the change of Asn52 to Asp and that had the ability to add a cysteine knob to β-subunit residues 92, 94, 95, and 96 was prepared (SEQ ID NO: 65). Co-expression of this analog in COS-7 cells with β-subunit analogs containing a cysteine at residues 92, 94, 95, and 96 led to the formation of acid-stable crosslinked heterodimers. The activities of these analogs and a chimera β-subunit analog that contained a cysteine in place of Arg96 (SEQ ID NO: 66) showed that the carboxyterminus of the α-subunit can be attached to several sites in the β-subunit, indicating that its location is not constrained unduely (FIG. 33). Further, the finding that the efficacy of the chimera analog was low showed that the conformation of this portion of the hormone has an influence on signal transduction.

One skilled in the art will readily appreciate that the present invention is well adapted to carry out the objects and obtain the ends and advantages mentioned as well as those inherent therein. The compositions along with the methods and procedures described herein are presently representative of preferred embodiments and are exemplary and not intended as limitations on the scope of the invention. Changes therein and other uses will occur to those skilled in the art which are encompassed within the spirit of the invention or defined by this scope with the claims.

It will be readily apparent to one skilled in the art that varying substitutions and modifications may be made to the invention disclosed herein without departing from the scope and spirit of the invention.

All patents and publications referenced herein are incorporated by reference to the same extent as if each individual publication was specifically and individually indicated to be incorporated by reference. The following list of references are likewise incorporated by reference.

### REFERENCES

1. Lapthom, A. J., Harris, D. C., Littlejohn, A., Lustbader, J. W., Canfield, R. E., Machin, K. J., Morgan, F. J. & Isaacs, N. W. (1994) Nature 369, 455-461

2. Wu, H., Lustbader, J. W, Liu, Y., Canfield, R. E. & Hendrickson, W. A. (1994) Structure 2, 545-558.

3. Suganuma, N., Matzuk, M. M. & Boime, I. (1989) J. Biol. Chem. 264, 19302-19307.

4. Wang, Y. H., Bemard, M. P. & Moyle, W. R. (2000) Mol. Cell. Endocinol. 170, 67-77.

5. Segaloff, D. L. & Ascoli, M. (1993) Endocr. Rev. 14, 324-347.

6. Kobe, B. & Deisenhofer J. (1993) Nature 366, 751-756.

7. Braun, T., Schofield, P. R. & Sprengel, R. (1991) EMBO. J. 10, 1885-1890.

8. Thomas, D., Rozell, T. G., Liu, X. & Segaloff, D. L. (1996) Mol. Endocrinol. 10, 760-768.

9. Bernard, M. P., Myers, R. V. & Moyle, W. R. (1998) Biochem. J. 335, 611-617.

10. Cosowsky, L., Rao, S. X. V., Macdonald, G. J., Papkoff, H., Campbell, R. K. & Moyle, W. R. (1995) J. Biol. Chem. 270, 20011-20019.

11. Moyle, W. R., Campbell, R. K, Rao, S. N. V., Ayad, N. G., Bernard, M. P., Han, Y. & Wang, Y. (1995) J. Biol. Chem. 270, 20020-20031.

12. Myers, R. V., Wang, Y. & Moyle, W. R. (2000) Biochim. Biophys. Acta 1475, 390-394.

13. Jiang, X., Dreano, M., Buckler, D. R., Cheng, S., Ythier, A., Wu, H., Hendrickson, W. A., Tayar, N. E. & el Tayar, N. (1995) Structure 3, 1341-1353.

14. Peng, K. C., Bousfield, G. R., Puett, D. & Ward, D. N. (1996) Journal of Protein Chemistry 15, 547-552.

15. Xia, H., Chen, F. & Puett, D. (1994) Endocrinol. 134, 1768-1770.

16. Pierce, J. G. & Parsons, T. F. (1981) Annu. Rev. Biochem. 50, 465-495.

17. Chen, F., Wang, Y. & Puett, D. (1992) Mol. Endocrinol. 6, 914-919.

18. Campbell, R. K., Dean Emig, D. M. & Moyle, W. R. (1991) Proc. Natl. Acad. Sci. (U.S.A.) 88, 760-764.

19. Campbell, R. K, Bergert, E. R.; Wang, Y., Morris, J. C. & Moyle, W. R. (1997) Nature Biotech. 15, 439-443

20. Grossmann, .M, Szkudlinski, M. W., Wong, R., Dias, J. A., Ji, T. H. & Weintraub, B. D. (1997) J. Biol. Chem. 272, 15532-15540.

21. Lindau-Shepard, B., Roth, K. E. & Dias, J. A. (1994) Endocrinol. 135, 1235-1240.

22. Moyle, W. R., Matzuk, M. M, Campbell, R. K, Cogliani, E., Dean Emig, D. M., Krichevsky, A., Barnett, R. W. & Boime, I. (1990) J. Biol. Chem. 265, 8511-8518.

23. Xing, Y., Lin, W., Jiang, M., Myers, R. V., Cao, D., Bernard, M. P. & Moyle, W. R. Alternatively folded choriogonadotropin analogs: implications for hormone folding and biological activity. Journal of Biological Chemistry. 2001.

24. Moyle, W. R., Ehrlich, P. H. & Canfield, R. E. (1982) Proc. Natl. Acad. Sci. (U.S.A.) 79, 2245-2249.

25. Moyle, W. R., Campbell, R. K., Myers, R. V., Bernard, M. P., Han, Y. & Wang, X. (1994) Nature 368, 251-255.

26. Matzuk, M. M. & Boime, I. (1988) J. Cell. Biol. 106, 1049-1059.

27. Einstein, M., Lin, W., Macdonald, G. J. & Moyle, W. R. (2001) Exp. Biol. Med. 226, 581-590.

28. Slaughter, S., Wang, Y. H., Myers, R. V. & Moyle, W. R. (1995) Mol. Cell. Endocrinol. 112, 21-25.

29. Yen, S. S. C, Llerena, O., Little, B. & Pearson, O. H. (1968) J. Clin. Endocrinol. Metab. 28, 1763-1767.

30. Yoo, J., Ji, I. & Ji, T. H. (1991) J. Biol. Chem. 266, 17741-17743.

31. Ji, I., Zeng, H. & Ji, T. H. (1993) J. Biol. Chem. 268, 22971-22974.

32. Han, Y., Bernard, M. P. & Moyle, W. R. (1996) Mol. Cell. Endocrinol. 124, 151-161.

33. Clackson, T. & Wells, J. A. (1995) Science 267, 383-386.

34. Wells, J. A. (1996) Proc. Natl. Acad. Sci. (U.S.A.) 83, 1-6.

35. Zlokarnik, G., Negulescu, P. A., Knapp, T. E., Mere, L., Burres, N., Feng, L., Whitney, M., Roemer, K. & Tsien, R. Y. (1998) Science 279, 84-88.

36. Moore, J. T., Davis, S. T. & Dev, I. K. (1997) Anal. Biochem. 247, 203-209.

37. Strynadka, N. C., Jensen, S. E., Johns, K., Blanchard, H., Page, M., Matagne, A., Frere, J. M. & James, M. N. (1994) Nature 368, 657-660.

What is claimed is:

1. A composition comprising,
a protein portion, wherein the protein portion contains a substituted cysteine residue at a desired location to be tagged;
a tail portion at the terminal end of the protein portion; and
a knob, wherein the knob is located at the free terminal end of the tail portion and contains a cysteine residue, and wherein the cysteine residue of the knob is capable of forming a disulfide with the substituted cysteine in the protein portion.

2. The composition of claim 1, wherein the tail contains a protease cleavage site.

3. The composition of claim 1, wherein the knob comprises an epitope tag.

4. The composition of claim 1, wherein the knob comprises a polypeptide.

5. The composition of claim 1, wherein the knob comprises a protein.

6. The composition of claim 5, wherein the knob cysteine is located on the surface of the protein.

7. The composition of claim 1, wherein the protein portion is a monomeric protein.

8. The composition of claim 1, wherein the protein portion is a multimeric protein.

9. A method for tagging a protein at a specific site comprising,
a) selecting a desired protein;
b) locating a specific site on the desired protein to be tagged;
c) selecting a desired knob, wherein the desired knob contains a cysteine residue;
d) preparing a construct encoding the desired protein, a tail portion and the desired knob, wherein the desired protein has a cysteine residue substituted at the site to be tagged;
e) inserting the construct into a cell for expression of the tagged protein, wherein the cysteine in the knob and the substituted cysteine in the desired protein form a disulfide bond.

10. The method of claim 9, wherein the knob comprises an epitope tag, a signal sequence or a protein.

11. The method of claim 10, wherein the knob protein is a protease.

12. The method of claim 9, wherein the desired protein is monomeric.

13. The method of claim 9, wherein the desired portein is multimeric.

14. A protein purification method comprising,
a) inserting a construct capable of expression in a cell, wherein the construct encodes a protein, wherein the protein comprises a cysteine residue substituted at a desired site to be tagged, a tail portion at the terminal end of the protein, wherein the tail portion contains a protease cleavage site, and a knob at the end of the tail portion, wherein the knob contains a cysteine residue;
b) lysing the cell;
c) purifying the protein based on the characteristics of the knob.

15. A method for tagging hCG comprising,
a) preparing a construct capable of expressing native HCGβ or HCGβ-S138C;
b) preparing a construct capable of expressing native hCGα or hCGα cysteine substituted analogs;
c) inserting the constructs of step a) and b) into COS-7 cells for co-expression.

16. The method of claim 15, wherein the construct of step a) further comprises fusing a protein to residue 140 or 145 of hCGβ.

17. The method of claim 16, wherein the protein is β-lactamase.

18. The method of claim 15, wherein the hCGα cysteine substituted analogs are selected from SEQ ID NO: 1 thru SEQ ID NO: 35.

19. A method for mapping the distance between protein molecules comprising,
a) selecting a first protein molecule;
b) selecting a second protein molecule, wherein the first protein molecule and the second protein molecule interact;
c) producing first protein molecules, wherein each first protein molecule produced contains a knob located at a different site on the first protein;
d) producing the second protein molecule;
e) using the proteins produced in steps c) and d) to analyze the distance between the first protein and the second protein.
